(19) United States
(12) Patent Application Publication
(10) Pub. No.: US 2007/0048293 A1
(43) Pub. Date: Mar. 1, 2007

(54) Manipulation of PTEN in T cells as a strategy to modulate immune responses

(75) Inventors: Laurence A. Turka, Merion Station, PA (US); Steven J. Bensinger, Los Angeles, CA (US); Patrick Walsh, Philadelphia, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Trustees of the University of Pennsylvania

(21) Appl. No.: 11/444,695

(22) Filed: May 31, 2006

Related U.S. Application Data
(60) Provisional application No. 60/685,837, filed on May 31, 2005.
(60) Provisional application No. 60/763,419, filed on Jan. 30, 2006.

(51) Int. Cl. A61K 48/00; C07H 21/02; C12N 5/08; A61K 9/127; C12N 15/88

(52) U.S. Cl. 424/93.21; 435/372; 435/458; 424/450; 536/23.1

(57) ABSTRACT

The present invention relates to compositions and methods for modulating the expression and/or activity of PTEN in T cells, regulatory T cells, and CD4 T cells. The manipulation of PTEN provides a means for regulating an immune response.

### CROSS REFERENCE TO RELATED APPLICATIONS

The present application claims priority under 35 U.S.C. § 119(e) to U.S. Provisional Application No. 60/685,837, filed on May 31, 2005, and U.S. Provisional Application No. 60/763,419, filed Jan. 30, 2006, each of which application is incorporated by reference herein in its entirety.

### STATEMENT REGARDING FEDERALLY SPONSORED RESEARCH OR DEVELOPMENT

This invention was made, in part, using funds obtained from the U.S. Government (National Institutes of Health Grant Nos. AI-37691, AI-41521, and AI-43626), and the U.S. Government may therefore have certain rights in this invention.

### BACKGROUND OF THE INVENTION

Interleukin-2 plays a central role in modulating immune responses, lymphocyte homeostasis, and tolerance induction of T lymphocytes (Waldmann et al., 2001, Immunity 14:105). The high-affinity IL-2R is a heterotrimeric complex composed of the α-chain (CD25), β-chain (CD122), and the common γ-chain (CD132). Engagement of the IL-2R on activated T cells initiates a complex signaling program that can induce proliferation, increase survival, as well as prime for activation-induced cell death (Refaeli et al., 1998, Immunity 8:615). More recently, studies on IL-2, CD25, and CD122 knockout mice have identified an essential role for IL-2 signals in the development and survival of CD4⁺CD25⁺ regulatory T cells (Tregs) (Malek et al., 2002, Immunity 17:167; Murakami et al., 2002, Proc. Natl. Acad. Sci. USA 99:8832; Almeida et al., 2002, J. Immunol. 169:4850).

CD4⁺CD25⁺ Tregs are a distinct population of T lymphocytes that have the capacity to dominantly suppress the proliferation of responder T cells in vitro and inhibit autoimmune disease in vivo (Sakaguchi et al., 1995, J. Immunol. 155:1151; Thornton et al., 1998, J. Exp. Med. 188:287). Despite expression of all three subunits of the high-affinity IL-2R, CD4⁺CD25⁺ T cells remain hypoproliferative when stimulated with IL-2 alone. However, combinations of signals, such as TCR and IL-2 or glucocorticoid-induced TNFR and IL-2, result in both the proliferation and the transient loss of suppressive characteristics (Takahashi et al., 1998, Int. Immunol. 10:1969; McHugh et al., 2002, Immunity 16:311; Shimizu et al., 2002, Nat. Immunol. 3:135). The molecular mechanism underlying these observations remains unknown.

The current signaling paradigm of IL-2-mediated proliferation and survival in activated T cells requires a coordinated effort between multiple signaling pathways downstream of the interleukin 2 receptor (IL-2R) (Brennan et al., 1997, Immunity 7:679; Moriggl et al., 1999, Immunity 10:249). The IL-2R has no intrinsic catalytic activity and relies on the ligand-mediated heterodimerization of the IL-2R to initiate activation of extrinsic signaling molecules, such as the Janus kinase (JAK). These initial events result in the subsequent activation of the transcription factor STAT5, as well as the recruitment of the phosphatidylinositol 3-kinase (PI3K) and Ras-mitogen-activated protein kinase (MAPK) signaling pathways (Waldmann et al., 2001, Immunity 14:105; Gaffen et al., 2001, Cytokine 14:63). This complex signaling system ultimately results in the up-regulation of genes that are critical for cell cycle progression and survival. Although IL-2 is required for the development and peripheral survival of CD4⁺CD25⁺ T cells, their hypoproliferative response to IL-2 in vitro suggests a differential pattern of IL-2R signaling compared with their activated T lymphocyte counterparts.

Naturally occurring CD25+CD4+ suppressor cells (Tregs) cells play an active part in establishing and maintaining immunological unresponsiveness to self constituents (i.e., immunological self tolerance) and negative control of various immune responses to non-self antigens. There is a paucity of reliable markers for defining Tregs, but naturally occurring CD25+CD4+ Tregs are the most widely studied because accumulating evidence indicates that this population plays a crucial role in the maintenance of immunological self tolerance and negative control of pathological as well as physiological immune responses. The natural presence of these cells in the immune system as a phenotypically distinct population makes them a good target for designing ways to treat or prevent immunological diseases and to control pathological as well as physiological immune responses. However, little, if any methods exist to expand and manipulate this population of cells. The present invention satisfies this need.

Furthermore, and also critical to T cell biology, the PI3K signaling pathway plays a central role in growth factor-mediated cellular functions such as proliferation, survival, growth, and glucose homeostasis. Activation of PI3K results in the production of 3′-phosphoinositide lipids, such as PIP₃ and phosphatidylinositol 4,5-bisphosphate (PIP2), the phosphorylation of which into phosphatidylinositol tris 3,4,5-phosphate (PIP3) is catalyzed by PI3K. Lipid second messengers, such as PIP2 and PIP3 bind to the plextrin homology domains of target proteins and directly recruit a wide variety of downstream effector molecules, such as Akt, PDK1, and Itk. Studies on both the IL-2R and CD28 have demonstrated that PI3K signaling and activation of Akt plays an important role in promoting T cell survival and proliferation (Brennan et al., 1997, Immunity 7:679; Van Parijs et al., 1999, Immunity 11:281; Kelly et al., 2002, J. Immunol. 168:597). Akt (protein kinase B) is a serine/threonine kinase that is a downstream target of PI3K signaling. Activation of Akt is thought to promote survival through a number of mechanisms including up-regulation of bcl-2 (Parsons et al., 2001, J. Immunol. 167:42), phosphorylation of proapoptotic bad, and its subsequent sequestration away from antiapoptotic bcl-2 family members (Datta et al., 1997, Cell 91:231) as well as negatively controlling the activity of proapoptotic Forkhead Family transcription factors (Tang et al., 1999, J. Biol. Chem. 274:16741).

The role of CD28 costimulation in T cell activation and T cell immune responses is well known. T cell receptor (TCR) stimulation alone only weakly (if at all) activates PI3K. CD28 costimulation, in large part via its ability to activate PI3K, synergizes with TCR ligation to allow T cells to respond to low concentrations of antigen, and even at optimally high concentrations of antigen, CD28 costimulation greatly augments T cell cytokine production and proliferation. In addition, T cell stimulation without CD28 costimulation induces T cells to become anergic, a state in which they are refractory to restimulation (as measured by failure to produce IL-2), even if CD28 is provided during the second stimulation.

The tumor suppressor gene PTEN (phosphatase and tensin analog on chromosome ten) is a lipid phosphatase that catalyzes the reverse reaction of PI3K, i.e. it converts PIP3 back to PIP2. In addition, PTEN is expressed at high levels in resting T cells, indicating that it might be part of the normal control mechanism that modulates T cell immunity.

CD4 T cells play a significant role in virtually every adaptive immune response. The presence of these cells is crucial to tumor immunity, infectious disease immunity, and regulating autoimmunity. Refined methods for the control of these cells leads to the means to treat or prevent immunological diseases and to control pathological as well as physiological immune responses. However, control of these cells at the molecular level remains elusive. The present invention also satisfies this need.

### BRIEF SUMMARY OF THE INVENTION

The present includes an isolated PTEN-deficient T cell, wherein the T cell is selected from the group consisting of a cytotoxic T cell, a helper T cell, and a regulatory T cell.

In one aspect, PTEN in the T cell is mutated.

In another aspect, PTEN in the T cell is deleted.

In yet another aspect, expression of PTEN in the T cell is inhibited using an inhibitor selected from the group consisting of a small interfering RNA (siRNA), a microRNA, an antisense nucleic acid, a ribozyme, an expression vector encoding a transdominant negative mutant, an intracellular antibody, a peptide and a small molecule.

Preferably, the inhibitor is an siRNA. The siRNA of the invention is selected from the group consisting of a double stranded oligonucleotide, a single stranded oligonucleotide, and a polynucleotide. In one aspect, the siRNA is chemically synthesized.

In another aspect, the inhibitor further comprises a physiologically acceptable carrier. Preferably, the physiologically acceptable carrier is a liposome.

In a further aspect, the inhibitor is encoded by an isolated polynucleotide cloned into an expression vector. Preferably, the expression vector is selected from the group consisting of a plasmid DNA, a viral vector, and a mammalian vector.

In one aspect, the expression vector further comprises an integration signal sequence which facilitates integration of the isolated polynucleotide into the genome of a host cell.

In another aspect, the PTEN deficient T cell is capable of regulating an immune response. Preferably, the immune response is associated with a disease selected from the group consisting of an infectious disease, a cancer, an autoimmune disease, graft rejection, atherosclerosis, and graft versus host disease (GVHD).

In yet another aspect, the PTEN deficient T cell exhibits an increased proliferation rate compared to an otherwise identical functional PTEN T cell.

The present invention includes a genetically modified T cell expressing an increased level of a PTEN polynucleotide compared with an otherwise identical T cell not so genetically modified. Preferably, T cell is selection from the group consisting of a cytotoxic T cell, a helper T cell, and a regulatory T cell.

In one aspect, PTEN polynucleotide is expressed from a vector selected from the group consisting of a plasmid DNA, a viral vector, and a mammalian vector.

The invention also includes a method of regulating T cell cellular proliferation comprising modulating PTEN expression and/or activity in a T cell by adding to the cell a composition that regulates the expression and or activity of PTEN.

In one aspect, the T cell is selection from the group consisting of a cytotoxic T cell, a helper T cell, and a regulatory T cell.

In another aspect, the expression of a PTEN polynucleotide in a T cell is down-regulated.

In yet another aspect, the expression of a PTEN polynucleotide in a T cell is increased.

The invention also encompasses a method of regulating an immune response comprising modulating PTEN expression and/or activity in the T cell by adding to the cell a composition that regulates the expression and or activity of PTEN.

In addition, the invention includes a method of treating a disease or disorder in a patient, wherein the disease or disorder is selected from the group consisting of an infectious disease, a cancer, an autoimmune disease, graft rejection, atherosclerosis, and graft versus host disease (GVHD). The method comprises administering to the patient an isolated PTEN-deficient T cell.

In one aspect, the T cell is selected from the group consisting of a cytotoxic T cell, a helper T cell, and a regulatory T cell.

In another aspect, the T cell is cultured in vitro prior to administering to the patient in need thereof.

In yet another aspect, the T cell is cultured in vitro in the presence of IL-2.

The invention also includes a method of treating a disease or disorder in a patient, wherein the disease or disorder is selected from the group consisting of an infectious disease, a cancer, an autoimmune disease, graft rejection, atherosclerosis, and graft versus host disease (GVHD). The method comprises administering a composition comprising an inhibitor of PTEN to a patient in need thereof, wherein the inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

The invention also includes a method of treating a disease or disorder in a patient, wherein the disease or disorder is selected from the group consisting of an infectious disease, a cancer, an autoimmune disease, graft rejection, atherosclerosis, and graft versus host disease (GVHD). The method comprising administering an isolated T cell so modified to prevent downregulation of PTEN to the patient.

In another embodiment, the invention includes a method of treating a disease or disorder in a patient, wherein the disease or disorder is selected from the group consisting of an infectious disease, a cancer, an autoimmune disease, graft rejection, atherosclerosis, and graft versus host disease (GVHD). The method comprising administering a composition comprising a polynucleotide encoding PTEN to a patient in need thereof.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A through 1D, is a series of charts demonstrating that IL-2 mediates the survival and cellular enlargement of CD4⁺CD25⁺ T cells in vitro.

FIG. 2, comprising FIGS. 2A through 2C, is a series of charts demonstrating that CD4⁺CD25⁺ T cells have a distinct IL-2R signaling that mediates survival independent of PI3K.

FIG. 3, comprising FIGS. 3A through 3C, is a series of charts demonstrating that IL-2R signaling mediates a distinct pattern of gene transcription and cell cycle progression in CD4⁺CD25⁺ T cells.

FIG. 4, comprising FIGS. 4A through 4C, is a series of charts demonstrating that IL-2 activates PI3K and negative regulation of PI3K signaling occurs downstream of PI3K.

FIG. 5, comprising FIGS. 5A through 5C, is a series of charts demonstration that activation of TCR down-regulates PTEN and restores IL-2-mediated PI3K signaling.

FIG. 6 is a chart demonstrating that manipulation of PTEN as a means to promote regulatory cell growth does not perturb the capacity of Tregs to suppress disease in vivo.

FIG. 7, comprising FIGS. 7A and 7B, is a series of charts demonstrating that inducible downregulation of PTEN is sufficient to confer IL-2 proliferative responsiveness on otherwise normal Tregs.

FIG. 8, comprising FIGS. 8A through 8C, is a series of charts demonstrating that PTEN constrains IL-2 responsiveness. Retroviral transduction of normal T cells with PTEN prevents the activation-induced downregulation of PTEN, and renders T cells unresponsive to IL-2.

FIG. 9, comprising FIGS. 9A through 9D, is a series of images depicting that PTEN deficient CD4+ T cells are hyper-responsive to TCR stimulation. FIG. 9A is a graph depicting IL-2 production as measured by ELISA in CD4+ T cells from the lymph nodes and spleen of wild-type and PTEN-ΔT mice stimulated with various concentrations of plate-bound anti-CD3. FIG. 9B is a series of histograms depicting the proliferation of CD4+ T cells of wild-type and PTEN-ΔT mice stimulated with various concentrations of plate-bound anti-CD3. FIG. 9C is a graph depicting lymph node-purified CD4+ T cell proliferation in iCre expressing and non-expressing mice after administration of tamoxifen and stimulation with plate-bound anti-CD3 alone or in combination with anti-CD28. FIG. 9D is a series of graphs depicting the viability at various time points of wild-type or PTEN deficient T cells stimulated as in FIG. 9C.

FIG. 10 is an image depicting that PTEN deficiency results in hyperactivation of the PI3K pathway in response to TCR signals. CD4+ T cells from wild-type and PTEN-ΔT mice were either unstimulated (UNS), or stimulated with anti-CD3 (αCD3), anti-CD28 (αCD28), or a combination (αCD3+αCD28). Cell lysates were probed with phosphospecific antibodies for Akt (p-Akt), GSK (p-GSK), and ERK (p-ERK). Membranes were re-probed with Akt or actin antibodies as loading controls.

FIG. 11, comprising FIGS. 11A and 11B, is a series of graphs depicting that activation of both PI3K and Akt is required for enhanced responsiveness of PTEN deficient T cells. The graphs depict IL-2 production as measured by ELISA in CD4+ T cells pre-incubated with either LY294002 (FIG. 11A) to inhibit PI3K activation, Triciribine (FIG. 11B) to inhibit Akt activity, or in the absence of chemical inhibitors, followed by stimulation with the indicated concentrations of anti-CD3.

FIG. 12, comprising FIGS. 12A and 12B, is a series of images depicting that CD4+ T cells lacing PTEN have a diminished requirement for costimulation. FIG. 12A is a series of histograms depicting the proliferation of CD4+ T cells stimulated with various concentrations of anti-CD3 alone or in combination with anti-CD28. FIG. 12B is a graph depicting flow cytometry quantification of CD4+ T cells from WT and PTEN-ΔT mice injected with splenocytes. Half of the mice also received an i.p. injection of CTLA4Ig.

FIG. 13 is a graph depicting that CD4+ T cells lacking PTEN are still responsive to Treg mediated suppression. FIG. 13 illustrates the proliferation, measured by ³H-thymidine incorporation, of wild type or PTEN deficient responder cells (CD4+CD25−CD45RB^{hi}) co-cultured at the indicated ratios with wild-type Tregs (CD4+ CD25+CD45RB^{lo}) stimulated in the presence of irradiated APCs with soluble anti-CD3.

FIG. 14, comprising FIGS. 14A through 14C, is a series of graphs depicting that PTEN regulates anergy induction both in vitro and in vivo. FIG. 14A is a graph depicting IL-2 production as measured by ELISA in wild-type and PTENΔT CD4+ T cells stimulated with anti-CD3 in the presence of APCs, either with or without the addition of CTLA4Ig, followed by restimulation with anti-CD3 and anti-CD28 coated beads. FIGS. 14B and 14C are a series of graphs depicting proliferation as measured by ³H-thymidine incorporation in Vβ8⁺ CD4⁺ T cells from wild-type and PTEN-ΔT mice injected with SEB after the isolated cells were restimulated with SEB.

FIG. 15, comprising FIGS. 15A-15G, is a series of images depicting the isolation and analysis of CD4+CD25+CD45RB^{lo} Tregs from PTEN-ΔT mice. FIG. 15A illustrates that PTEN is expressed at equivalent levels in CD4+CD25− and CD4+CD25+ T cell subsets from normal mice. FIG. 15B illustrates specific recombination at the Pten locus in the presence of Cre was shown by PCR amplification of a 849 bp product in genomic DNA isolated from CD4+ T cells from Cre−ve (wild type), Pten^{flox/+}Cre+ (PTEN het) and Pten^{flox/flox/}Cre+ (PTEN-ΔT) littermates. FIG. 15C illustrates expression of PTEN protein in CD4+ T cells isolated as above. β-Actin was used as a loading control. FIG. 15D illustrates expression of the T cell activation marker CD69 on CD4+ T cells from PTEN-ΔT mice and wild type littermates at 2 weeks, before the onset of disease, and at 8 weeks. FIG. 15E illustrates the frequency of CD4+ T cells which are CD25^{hi}CD45RB^{lo} from 2-week old PTEN-ΔT and wild type littermate mice. FIG. 15F illustrates real time PCR analysis of Foxp3 expression on FACS purified CD4+CD25−CD45RB^{hi} and CD4+CD25+CD45RB^{lo} cells from 2 week old PTEN-ΔT and wild type littermate mice. FIG. 15G illustrates how purified CD4+CD25−CD45RB^{hi} cells (1×10⁵) from littermate control mice were stimulated with anti-CD3 (0.5 μg/ml) plus irradiated APC's (3×10⁶) for 72 hours in the presence of the indicated ratios of CD4+CD25+CD45RB^{lo} Tregs purified from either PTEN-ΔT mice or wild type mice. Tritiated thymidine was added to cultures for the final 16 hours before harvesting. All data are representative of at least two independent experiments.

FIG. 16, comprising FIGS. 16A-16B, depicts thymic development of CD4+Foxp3+ Tregs in the absence of PTEN. FIG. 16A illustrates that CD4+CD8+ double positive, CD4+ single positive and peripheral CD4+ T cells were isolated by FACS from 3-week old wild type and PTEN-ΔT mice. Cells were subsequently lysed and analyzed for expression of PTEN by immunoblotting. FIG. 16B illustrates that T depleted bone marrow cells from wild-type (Thy1.1+) and PTEN-ΔT (Thy1.2+) mice were used to reconstitute lethally irradiated Thy1.1+hosts. Mice were reconstituted with either 100% wild-type, 100% PTEN-ΔT or with 50%/50% mixture. Thymic regulatory subsets (CD4SP Foxp3+) were analyzed 10 weeks after reconstitution. Data shown are representative of results from 3 chimeric mice per condition.

FIG. 17, comprising FIGS. 17A-17F, depicts the proliferation of PTEN-ΔT CD4+CD25+CD45RB^{lo} Tregs in response to IL-2R stimulation. FIG. 17A illustrates that purified CD4+CD25+CD45RB^{lo} and CD4+CD25+CD45RB^{hi} cells from both wild-type and PTEN-ΔT mice were cultured at a constant density of 1×10⁵/well in the presence of rIL-2 (100 U/ml) for a 2 week period. At the indicated time points total cell numbers were quantitated by trypan blue exclusion. Data is representative of 5 different experiments. FIG. 17B illustrates that purified CD4+CD25+CD45RB^{lo} cells from wild type, PTEN-het and PTEN-ΔT mice were cultured in the presence of rIL-2 (100 U/ml) for 48 hours. Tritiated thymidine was added to cultures for the final 16 hours before harvesting. FIG. 17C illustrates that PTEN-ΔT CD4+CD25+CD45RB^{lo} cells were CFSE labeled and cultured in the presence of rIL-2 (100 U/ml) for 10 days. CFSE dilution was analyzed at the indicated time points by FACS analysis. Results are representative of four separate experiments. FIG. 17D illustrates that purified wild type and PTEN-ΔT CD4+CD25+CD45RB^{lo} cells and pre activated CD4+ T cell blasts were stimulated with titrated doses of rIL-2 as shown for 72 hrs. Tritiated thymidine was added to cultures for the final 16 hours before harvesting. FIG. 17E illustrates that CD4+ T cells were isolated from ER-Cre⁺/Pten^{flox/flox} mice and lysed immediately or after culture in the presence of rIL-2 (100 U/ml) and 4-OHT (1 nM). Samples were electrophoresed on an SDS PAGE gel, transferred to nitrocellulose membranes and probed as indicated. FIG. 17F illustrates that purified ER-Cre⁺/Pten^{flox/flox} CD4+CD25+ regulatory T cells were CFSE labeled and cultured in the presence of rIL-2 (100 U/ml) and 4-OHT (1 nM) for 7 days. CFSE dilution was analyzed by FACS. Results are representative of three independent experiments.

FIG. 18 is a series of images depicting re-expression of PTEN in PTEN-ΔT Tregs restores hypoproliferative response to IL-2. Purified PTEN-ΔT CD4+CD25+CD45RB^{lo} cells were CFSE labeled and retrovirally transduced as described in Materials and Methods with either MIGR1-NGFR empty vector (ev-NGFR) or PTEN-containing virus (PTEN-NGFR). Cells were analyzed for expression of human NGFR 96 hours after infection and CFSE dilution of NGFR positive cells analyzed by flow cytometry. Results are representative of 3 independent experiments.

FIG. 19, comprising FIGS. 19A-19C, depicts that PTEN-ΔT CD4+CD25+CD45RB^{lo} cells remain hypoproliferative to TCR stimulation. FIG. 19A illustrates that purified PTEN-ΔT or wild type CD4+CD25+CD45RB^{lo} cells were CFSE labeled and stimulated with plate bound anti-CD3 (5 μg/ml) for 72 hours. FIG. 19B illustrates that supernatants were harvested from the conditions described above after 24 hours stimulation, and levels of IL-2 were determined by ELISA. Data shown are the mean+/−SD of triplicate samples. Data is representative of three separate experiments. FIG. 19C illustrates that purified PTEN-ΔT or wild type CD4+CD25+CD45RB^{lo} cells were stimulated with IL-2 (100 U/ml), irradiated APCs and varying doses of anti-CD3 (2C11) as shown for 72 hours. Tritiated thymidine was added to cultures for the final 16 hours before harvesting. Data shown are the mean+/−SD of triplicate samples.

FIG. 20, comprising FIGS. 20A-20C, include a series of images depicting that PTEN-ΔT Tregs exhibit enhanced homeostatic expansion in the periphery. PTEN-ΔT mice and littermate controls were administered 1 mg BrdU every 12 hours for 3 days, at which time they were sacrificed and thymus and spleen cells were stained for BrdU. FIG. 20A illustrates that levels of BrdU incorporation in CD4+CD8-CD25+ and CD4+CD8-CD25− thymocytes. FIG. 20B illustrates that levels of BrdU incorporation in CD4+CD25+ and CD4+CD25− splenocytes. FIG. 20C illustrates the percentage BrdU positive CD4+CD25+ and CD4+CD25− splenocytes from PTEN-ΔT mice (n=6) and littermate controls (n=6).

FIG. 21 is a series of images depicting that deletion of PTEN facilitates IL-2R signaling downstream of PI-3kinase in Tregs. FACS purified CD4+CD25+CD45RBlo cells from PTEN-ΔT mice were expanded in the presence of rIL-2 (100 U/ml) for 8 days. Cells were washed extensively and along with freshly isolated wild type Tregs, were rested overnight in complete media. Cells were subsequently left untreated or stimulated with 100 U/ml rIL-2 for 30 mins. Samples were lysed and electrophoresed on an SDS PAGE gel, transferred to nitrocellulose membranes and probed as indicated.

FIG. 22, comprising FIGS. 22A-22E, include a series of images depicting that re-expression of PTEN in activated CD4+ T cells inhibits IL-2 mediated proliferation. FIG. 22A illustrates that splenocytes from DO11.10 TCR transgenic mice were stimulated with ova peptide (1 μg/ml). CD4+ T cells were purified by magnetic bead separation at the indicated time points and subsequently lysed. Samples were analysed for PTEN expression by western blot and membranes were stripped and reprobed for β-Actin as a loading control. Result is representative of three independent experiments. FIG. 22B illustrates that purified CD4+ T cells were retrovirally transduced, as described in Materials and Methods, with either MIGR1-NGFR empty vector (ev-NGFR) or PTEN-containing virus (PTEN-NGFR). Cells were analyzed for expression of human NGFR 48 hours after infection. Data shown illustrates typical transduction efficiencies achieved using these vectors. The gate drawn shows NGFR positive subsets used for comparison in subsequent experiments. FIG. 22C illustrates that NGFR positive cells were purified either by FACS sort or magnetic bead separation and cultured in the presence or absence of rIL-2 (20 U/ml) for 48 hours. Tritiated thymidine was added to cultures for the last 16 hours before harvesting. Data shown is the mean+/−SD of triplicate cultures and are representative of four independent experiments. FIG. 22D illustrates that cells as in panel C were analyzed for viability by 7-AAD incorporation. Data shown is representative of four independent experiments. FIG. 22E illustrates that purified CD4+ T cells were CFSE labeled before stimulation and retroviral transduction. After infection cells were cultured in the presence of rIL-2 (10 U/ml) for 48 hours and cells expressing identical levels of NGFR were analyzed for CFSE dilution by flow cytometry.

FIG. 23, comprising FIGS. 23A-23D, includes a series of images depicting the ex-vivo expansion of PTEN-ΔT Tregs does not affect their regulatory phenotype. FIG. 23A illustrates that real time PCR analysis of expression levels of Foxp3 mRNA in PTEN-ΔT CD4+CD25+CD45RB^{lo} cells, which have been expanded for 8 days ex vivo with rIL-2, compared to freshly isolated CD4+CD25+CD45RB^{lo} and CD4+CD25−CD45RB^{hi} cells from wild type littermate mice. Results are representative of three independent experiments. FIG. 23B illustrates that expression of Foxp3 protein on CFSE labeled PTEN-ΔT CD4+CD25+CD45RB^{lo} cells expanded for 8 days with rIL-2 (100 U/ml). FIG. 23C illustrates that PTEN-ΔT Tregs were expanded in the presence of rIL-2 for 8 days and subsequently washed extensively before co culture at the indicated ratio with wild type CD4+CD25−CD45RB^{hi} responder cells expressing the Thy1.1 congenic marker. Freshly isolated CD4+CD25+CDRB^{lo} cells from wild type mice were used for direct comparison. Cells were stimulated in the presence of 3×10⁵ irradiated APCs with soluble anti-CD3 (0.5 μg/ml) for 72 hours at which time CFSE dilution of Thy1.1 expressing responder cells was examined by flow cytometry. FIG. 23D illustrates the quantitative comparison of level of suppression by wild-type and PTEN-ΔT Tregs through calculation of number of mitotic events of Thy 1.1+responder cells.

FIG. 24, comprising FIGS. 24A-24B, is a series of images depicting the prevention of colitis by ex vivo expanded PTEN-ΔT Tregs. Rag 1^{-/-} mice were transferred with 6×10⁶ freshly isolated wild type CD4+CD25−CD45RB^{hi} cells either alone (n=5) or together with 3×10⁶ freshly isolated wild type Tregs (n=5) or PTEN-ΔT Tregs which had been expanded for 5 days ex vivo with rIL-2 (n=7). FIG. 24A illustrates that body weight is represented as percentage of initial weight 8 weeks after transfer. Statistical analysis performed using a paired students t-test. FIG. 24B illustrates the severity of colitis was histologically scored as described previously.

FIG. 25 is a series of images depicting that PTEN deficiency does not affect IL-2 mediated survival of Tregs. Freshly isolated wild-type or PTEN-ΔT Tregs were cultured for 24 hours in the presence or absence of rIL-2 (100 U/ml) and cell survival measured by staining with the vital dye 7-AAD followed by flow cytometric analysis.

FIG. 26 is an image depicting that PTEN-ΔT CD4+CD25−CD45RB^{hi} cells do not prevent colitis. Rag1^{-/-} mice were transferred with 6×10⁶ freshly isolated wild type CD4+CD25−CD45RB^{hi} cells either alone (n=5) or together with 3×10⁶ freshly isolated wild type Tregs (n=5) or PTEN-ΔT CD4+CD25−CD45RB^{hi} cells (n=5, one mouse died during the examination period). Body weight is represented as percentage of initial weight 8 weeks after transfer.

### DETAILED DESCRIPTION

The present invention relates to the discovery that CD4⁺CD25⁺ T cells (otherwise known as regulatory T cells; “Tregs”) have a distinct interleukin-2 receptor (IL-2R) signaling pattern. The present disclosure demonstrates that engagement of the IL-2R on Tregs results in the activation of the JAK/STAT signaling pathway, but fails to activate downstream targets of the PI3K signaling pathway, such as Akt or p70^{s6kinase}. Examination of IL-2-dependent PI3K signaling in CD4⁺CD25⁺T cells demonstrated that negative regulation of the PI3K signaling pathway is inversely associated with expression of the lipid phosphatase and tensin homologue deleted on chromosome 10 (PTEN). As such, the present invention includes compositions and methods for regulating T cell development and survival of T cells, particularly the development and survival of Tregs, without inducing a significant mitogenic response.

The present invention is also based on the discovery that non-regulatory T cells express PTEN, and that manipulating PTEN in these effector T cells (CD4 T cells) can be used as a means to control the responses of these T cells. Stemming from this discovery, the present invention encompasses the manipulation of PTEN to regulate, modulate and augment T cell-mediated responses.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Allogeneic” refers to a graft derived from a different animal of the same species.

“Antisense” refers particularly to the nucleic acid sequence of the non-coding strand of a double stranded DNA molecule encoding a polypeptide, or to a sequence which is substantially homologous to the non-coding strand. As defined herein, an antisense sequence is complementary to the sequence of a double stranded DNA molecule encoding a polypeptide. It is not necessary that the antisense sequence be complementary solely to the coding portion of the coding strand of the DNA molecule. The antisense sequence may be complementary to regulatory sequences specified on the coding strand of a DNA molecule encoding a polypeptide, which regulatory sequences control expression of the coding sequences.

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual to which it is later to be re-introduced into the individual.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “DNA” as used herein is defined as deoxyribonucleic acid.

“Donor antigen” refers to an antigen expressed by the donor tissue to be transplanted into the recipient.

“Recipient antigen” refers to a target for the immune response to the donor antigen.

As used herein, an “effector cell” refers to a cell which mediates an immune response against an antigen. An example of an effector cell includes, but is not limited to a T cell and a B cell.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “heterologous” as used herein is defined as DNA or RNA sequences or proteins that are derived from the different species.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the term “modulate” is meant to refer to any change in biological state, i.e. increasing, decreasing, and the like.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “polypeptide” as used herein is defined as a chain of amino acid residues, usually having a defined sequence. As used herein the term polypeptide is mutually inclusive of the terms “peptide” and “protein”.

“Proliferation” is used herein to refer to the reproduction or multiplication of similar forms of entities, for example proliferation of a cell. That is, proliferation encompasses production of a greater number of cells, and can be measured by, among other things, simply counting the numbers of cells, measuring incorporation of ³H-thymidine into the cell, and the like.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are culture in vitro. In other embodiments, the cells are not cultured in vitro.

The term “T-cell” as used herein is defined as a thymus-derived cell that participates in a variety of cell-mediated immune reactions.

As used herein, a “therapeutically effective amount” is the amount of a therapeutic composition sufficient to provide a beneficial effect to a mammal to which the composition is administered.

The phrase “under transcriptional control” or “operatively linked” as used herein means that the promoter is in the correct location and orientation in relation to a polynucleotide to control the initiation of transcription by RNA polymerase and expression of the polynucleotide.

The term “vaccine” as used herein is defined as a material used to provoke an immune response after administration of the material to a mammal.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “virus” as used herein is defined as a particle consisting of nucleic acid (RNA or DNA) enclosed in a protein coat, with or without an outer lipid envelope, which is capable of replicating within a whole cell.

“Xenogeneic” refers to a graft derived from a mammal of a different species.

### DESCRIPTION

The invention relates to the identification of a novel mechanism by which CD4⁺CD25⁺ T cells respond to IL-2 signals. Tregs exhibit a distinct pattern of IL-2R signaling in which the Janus kinase/STAT pathway remains intact, and IL-2 does not activate downstream targets of phosphatidylinositol 3-kinase (PI3K). Negative regulation of PI3K signaling and IL-2-mediated proliferation of CD4⁺CD25⁺ T cells is inversely associated with expression of the phosphatase and tensin homologue deleted on chromosome 10 (PTEN).

Based on the present disclosure, T cell development and survival can be regulated by manipulating PTEN in the cell. As such, the present invention includes compositions and methods for modulating the expression and/or activity of PTEN in a cell to regulate proliferation of the cell. The composition of the present invention is useful in providing a therapeutic benefit in cell therapy and/or vaccination.

In an embodiment of the invention, a PTEN-deficient Treg exhibits a enhanced proliferation rate compared with an otherwise identical Treg. As such, PTEN-deficient Tregs can be expanded in vitro and administered to a patient in need thereof. PTEN-deficient Tregs are immunologically functional, for example, they are capable of suppressing a disease in vivo (i.e. an autoimmune disease). The present invention further includes compositions and methods for modulating the expression and/or activity of PTEN in a CD4 T cell to regulate proliferation, activation and anergy in the cell.

In another embodiment, a polynucleotide encoding PTEN can be introduced to a normal T cell. Introduction of PTEN into a cell prevents downregulation of PTEN and renders the cell unresponsive to IL-2.

The present invention further relates to the identification of a novel mechanism by which CD4⁺ T cells respond to TCR stimulation in the absence of CD28 costimulation. Further, the present invention describes methods to reduce and/or inhibit the T cell anergy induced by TCR stimulation alone. As demonstrated by the data disclosed herein, deletion of PTEN is associated with induction of detectable PI3K activity, thus serving as a substitute for CD28 costimulation.

As demonstrated by the data disclosed herein, PI3K is activated downstream of a number of different receptors expressed on the surface of T cells, and is critical for regulating cell survival, proliferation, and chemotaxis. The phosphatase PTEN is a key negative regulator of PI3K activity.

The present data demonstrates CD4 T cells from mammals with a deletion of PTEN targeted to T cells, exhibit augmented responses to sub maximal anti-CD3 stimulation alone, comparable to what is observed in wild type CD4 T cells that also receive CD28 costimulation. Further, the present data strikingly demonstrate that anti-CD3 stimulation of PTEN-ΔT T cells fails to induce anergy even in the absence of CD28 costimulation. These data demonstrate that positive and negative regulation of PI3K by CD28 and PTEN, respectively, shape the fate of CD4 T cells following TCR stimulation.

In an embodiment of the invention, a PTEN-deficient T cell exhibits a heighten activation and/or proliferation rate compared with an otherwise identical T, especially in the absence of CD28 costimulation. As such, PTEN-deficient CD4 T cells can be expanded in vitro and administered to a patient in need thereof, without the need for costimulation. PTEN-deficient T cells are immunologically functional, as evidenced by the data disclosed herein, which demonstrates proliferation and IL-2 production in such cells.

The present invention is useful in immunotherapy, such as in immune adjuvants and cancer immunotherapy. That is, the present invention comprises methods to enhance the response of a T cell to an antigen or a disease by inhibiting PTEN. This is because, as demonstrated herein, manipulating PTEN does not require antigen specificity.

In another embodiment of the present invention, the activity of PTEN can be sustained or increased to increase T cell lethargy to block an immune response, such as in autoimmune disease or in transplantation.

Therefore, the present invention encompasses methods to manipulate T cells to increase proliferation and/or activation in the absence of costimulatory signals. Further, as demonstrated by the data disclosed herein, the present invention comprises methods for preventing the induction of anergy in a T cell. Further still, as demonstrated by the data disclosed herein, the drug CTLA4Ig, which blocks T cell costimulation, is ineffective in preventing immune responses in mice whose T cells lack PTEN, when compared with wild-type mice.

Regulation of PTEN

Based on the disclosure herein, the present invention comprises modulating PTEN expression and/or activity in a cell. Generating a PTEN deficient Treg provides a means to promote cellular proliferation. Loss of PTEN does not alter the ability of Tregs to prevent diseases in an in vivo model. Thus, manipulation of PTEN, for example inhibiting PTEN in a cell, offers a strategy to modulate an immune response.

Further based on the disclosure herein, the present invention comprises a method for modulating PTEN expression and/or activity in a cell, whereby generating a PTEN deficient T cell provides a means to promote cellular proliferation and/or activation. Loss of PTEN does not alter the ability of T cells to function normally. Thus, the invention provides a further method for manipulation of PTEN, as well as a strategy to modulate an immune response.

PTEN can be inhibited in a cell using a composition selected from the group consisting of a small interfering RNA (siRNA), a microRNA, an antisense nucleic acid, a ribozyme, an expression vector encoding a transdominant negative mutant, an intracellular antibody, an antibody that specifically binds the PTEN gene product, a peptide and a small.

One skilled in the art will appreciate, based on the disclosure provided herein, that one way to decrease the mRNA and/or protein levels of PTEN in a cell is by reducing or inhibiting expression of the nucleic acid encoding PTEN. Thus, the protein level of PTEN in a cell can also be decreased using a molecule or compound that inhibits or reduces gene expression such as, for example, an siRNA, an antisense molecule or a ribozyme.

An siRNA is an RNA molecule comprising a set of nucleotides that is targeted to a gene or polynucleotide of interest. As used herein, the term “siRNA” encompasses all forms of siRNA including, but not limited to (i) a double stranded RNA polynucleotide, (ii) a single stranded polynucleotide, and (iii) a polynucleotide of either (i) or (ii) wherein such a polynucleotide, has one, two, three, four or more nucleotide alterations or substitutions therein.

Based on the present disclosure, it should be appreciated that the siRNAs of the present invention may effect the target polypeptide expression to different degrees. The siRNAs thus must first be tested for their effectiveness. Selection of siRNAs are made therefrom based on the ability of a given siRNA to interfere with or modulate the expression of the target polypeptide.

One skilled in the art will readily appreciate that as a result of the degeneracy of the genetic code, many different nucleotide sequences may encode the same polypeptide. That is, an amino acid may be encoded by one of several different codons, and a person skilled in the art can readily determine that while one particular nucleotide sequence may differ from another, the polynucleotides may in fact encode polypeptides with identical amino acid sequences. As such, polynucleotides that vary due to differences in codon usage are specifically contemplated by the present invention.

In yet another embodiment, the expression of PTEN can be inhibited using an antisense nucleic acid sequence. Preferably, the antisense nucleic acid is expressed by a plasmid vector. The antisense expressing vector is used to transfect a mammalian cell or the mammal itself, thereby causing reduced endogenous expression PTEN in the cell. However, the invention should not be construed to be limited to inhibiting expression of PTEN by transfection of cells with antisense molecules. Rather, the invention encompasses other methods known in the art for inhibiting expression or activity of a protein in the cell including, but not limited to, the use of a ribozyme, the expression of a non-functional PTEN (i.e. transdominant negative mutant) and use of an intracellular antibody.

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In another aspect of the invention, PTEN can be inhibited by way of inactivating and/or sequestering PTEN in a cell. As such, inhibiting the effects of PTEN can be accomplished by using a transdominant negative mutant. Alternatively an intracellular antibody specific for PTEN, otherwise known as an antagonist to PTEN, may be used. In one embodiment, the antagonist is a protein and/or compound having the desirable property of interacting with a binding partner of PTEN and thereby competing with the corresponding wild-type PTEN. In another embodiment, the antagonist is a protein and/or compound having the desirable property of interacting with PTEN and thereby sequestering PTEN.

Vectors

The invention includes an isolated nucleic acid encoding an inhibitor of PTEN, operably linked to a nucleic acid comprising a promoter/regulatory sequence such that the nucleic acid is preferably capable of directing expression of the protein encoded by the nucleic acid. In other related aspects, the invention includes an isolated nucleic acid encoding PTEN. The present disclosure demonstrates that retroviral transduction of PTEN into a T cell prevents the downregulation of PTEN, and therefore renders the T cell unresponsive to IL-2. The present invention also provides that induction of PTEN in a T cell can be used as a means to block immune responses and is therefore useful in the treatment of autoimmune diseases and transplant rejection.

The invention also encompasses expression vectors and methods for the introduction of exogenous DNA into cells with concomitant expression of the exogenous DNA in the cells. The incorporation of a desired polynucleotide into a vector and the choice of vectors is well-known in the art as described in, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

The polynucleotide of the invention can be cloned into a variety of vectors. However, the present invention should not be construed to be limited to any particular vector. Instead, the present invention should be construed to encompass a wide plethora of vectors which are readily available and/or well-known in the art. For example, an the polynucleotide of the invention can be cloned into a vector including, but not limited to a plasmid, a phagemid, a phage derivative, a mammal virus, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

In the context of an expression vector, the vector can be readily introduced into a host cell, e.g., mammalian, bacterial, yeast or insect cell by any method in the art. For example, the expression vector can be transferred into a host cell by physical, chemical or biological means. It is readily understood that the introduction of the expression vector comprising the polynucleotide of the invention yields a silenced cell with respect to a cytokine signaling regulator.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell or otherwise expose a cell to the polynucleotide of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In the case where a non-viral delivery system is utilized, a preferred delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

Therapeutic Application

In one embodiment, the invention includes a vaccine. Preferably, the vaccine is a cellular vaccine, whereby a cell may be isolated from a culture, tissue, organ or organism and administered to a mammal in need thereof. The cell may also express one or more additional vaccine components, such as immunomodulators or adjuvants. In a preferred embodiment, the cellular vaccine of the present invention comprises a PTEN deficient human T cell and in a more preferred embodiment, the T cell is a regulator T cell (Treg) or a CD4⁺CD25⁻ T cell. In another embodiment, the cell has been transduced to express PTEN to prevent downregulation of PTEN.

In another embodiment, the cellular vaccine comprises a Treg that has been manipulated according to the present invention to acquire loss of PTEN. The PTEN deficient cell can be cultured in vitro to expand the number of Tregs sufficient for therapeutic and/or experimental use. A benefit of generating a PTEN deficient Treg is that loss of PTEN does not perturb the capacity of the cells to suppress a disease in vivo. Yet the loss of PTEN allows for the rapid expansion of Tregs. Based on the disclosure herein, PTEN deficient Tregs exhibit a heighten proliferation rate compare to an otherwise identical Treg.

In yet another embodiment, the cellular vaccine comprises a T cell that has been manipulated according to the present invention to inhibit the expression and/or activity of PTEN. The PTEN deficient cell can be cultured in vitro to expand the number of T cells sufficient for therapeutic and/or experimental use. A benefit of generating a PTEN deficient T cell is that loss of PTEN enhance the immune response to a particular antigen, but is not an antigen specific immune system stimulation. Further, the loss of PTEN allows for rapid expansion of T cells. Based on the disclosure herein, PTEN deficient T cells exhibit a heightened proliferation rate compare to an otherwise identical T cell comprising intact or fully active PTEN.

Ex vivo procedures are well known in the art and are discussed more fully below. Briefly, cells are isolated from a mammal (preferably a human) and activated (i.e., transduced or transfected in vitro) with a vector expressing a polynucleotide of the present invention. The cell can then be administered to a mammalian recipient to provide a therapeutic benefit. The mammalian recipient may be a human and the cell so modified can be autologous with respect to the recipient. Alternatively, the cells can be allogeneic, syngeneic or xenogeneic with respect to the recipient.

The procedure for ex vivo expansion of hematopoietic stem and progenitor cells described in U.S. Pat. No. 5,199,942, incorporated herein by reference, can be applied to the cells of the present invention. Other suitable methods are known in the art, therefore the present invention is not limited to any particular method of ex vivo expansion of the cells.

In any event, the T cells expanded according to the present invention are administered to a mammal. The amount of cells administered can range from about 1 million cells to about 300 billion. The cells may be infused into the mammal or may be administered by other parenteral means. The mammal is preferably a human patient in need thereof. The precise dosage administered will vary depending upon any number of factors, including but not limited to, the type of mammal and type of disease state being treated, the age of the mammal and the route of administration.

The cell may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

A T cell (or cells expanded thereof) may be co-administered to the mammal with the various other compounds (cytokines, chemotherapeutic and/or antiviral drugs, among many others). Alternatively, the compound(s) may be administered an hour, a day, a week, a month, or even more, in advance of the T cell (or cells expanded thereby), or any permutation thereof. Further, the compound(s) may be administered an hour, a day, a week, or even more, after administration of T cell (or cells expanded thereby), or any permutation thereof. The frequency and administration regimen will be readily apparent to the skilled artisan and will depend upon any number of factors such as those already discussed elsewhere herein.

In addition to using a cell-based vaccine in terms of ex vivo immunization, the present invention also provides compositions and methods for in vivo immunization to regulate an immune response in a mammal.

With respect to in vivo immunization, a T cell can be manipulated to either 1) inhibit, or 2) induce expression and/or activity of PTEN. Preferably, the T cell is a regulatory T cell (Treg). Based on the present invention, loss of PTEN in Tregs increases the proliferation rate of the cell, but does not perturb the biological function of the Treg. For example, inhibition of PTEN in a Treg does not perturb the capacity of the Treg to suppress a disease in vivo. With respect to manipulation of a T cell to induce expression of PTEN, or otherwise prevent downregulation of PTEN, the present disclosure demonstrates that such a T cell is rendered unresponsive to IL-2.

Additionally, with respect to in vivo immunization according to the invention, a T cell can be manipulated to either 1) inhibit, or 2) induce expression and/or activity of PTEN, wherein the T cell is a CD4⁺CD25⁻ T cell. Based on the present invention, loss of PTEN in T cells increases the proliferation rate of the cell, but does not perturb the biological function of the T cell. Furthermore, T cells in which PTEN has been deleted are not induced to anergy by TCR stimulation alone.

The invention also encompasses the use of pharmaceutical compositions of an appropriate protein or peptide and/or isolated nucleic acid to practice the methods of the invention.

The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs, birds including commercially relevant birds such as chickens, ducks, geese, and turkeys, fish including farm-raised fish and aquarium fish, and crustaceans such as farm-raised shellfish.

Pharmaceutical compositions that are useful in the methods of the invention may be prepared, packaged, or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers and AZT, protease inhibitors, reverse transcriptase inhibitors, interleukin-2, interferons, cytokines, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### EXAMPLES

The invention is now described with reference to the following Examples. These Examples are provided for the purpose of illustration only, and the invention is not limited to these Examples, but rather encompasses all variations which are evident as a result of the teachings provided herein.

Despite expression of the high-affinity interleukin 2 receptor (IL-2R), CD4⁺CD25⁺ regulatory T cells (Tregs) are hypoproliferative upon IL-2R stimulation in vitro. However the mechanisms by which CD4⁺CD25⁺ T cells respond to IL-2 signals are undefined. Given the central role of IL-2 in the biology of T cells, the following experiments were designed to characterize the cellular and molecular responses of these cells to IL-2R signals. The results presented herein demonstrate that CD4⁺CD25⁺ T cells have a distinct IL-2R signaling pattern. It was observed that engagement of the IL-2R on Tregs resulted in the activation of the JAK/STAT signaling pathway, but failed to activate downstream targets of the PI3K signaling pathway, such as Akt or p70^{s6kinase}. This failure to activate PI3K/Akt did not abrogate the antiapoptotic effect of IL-2 on Tregs. Examination of IL-2-dependent PI3K signaling in CD4⁺CD25⁺ T cells revealed that negative regulation of the PI3K signaling pathway is inversely associated with expression of the lipid phosphatase and tensin homologue deleted on chromosome 10 (PTEN). Taken together, the results presented herein demonstrate a unique IL-2R signaling pattern, which defines how IL-2 mediates the development and survival of CD4⁺CD25⁺ T cells without inducing a significant mitogenic response.

The materials and methods employed in the experiments disclosed herein are now described.

Mice

BALB/c mice were purchased from The Jackson Laboratory (Bar Harbor, Me.) and maintained under specific pathogen-free conditions. Mice with a targeted floxed PTEN allele were obtained from the University of Toronto, and were crossed with CD4-Cre mice. The mice obtained from this cross were called PTEN-ΔT mice. CD4-Cre mice were obtained from the University of Pennsylvania.

As T cell specific deficiency of PTEN results in early lethality, Pten^{flox/+} CD4-Cre⁺ mice were bred with Pten^{flox/flox} mice to generate offspring. Mice were genotyped as described and offspring, which were found to express the Cre transgene and were homozygous for the Pten^{flox} allele were used for analysis as homozygous mutant mice (PTEN-ΔT) while Cre negative littermates were used as controls. Cre-ER mice have been described previously and were crossed with Pten^{flox/flox} mice and genotyped as described above. Rag1^{-/-} mice, C57BL/6 mice expressing the Thy1.1 congenic marker and DO11.10 TCR transgenic mice were purchased from The Jackson Laboratory (Bar Harbor, Me.). All colonies were maintained under SPF conditions at the animal facilities of the University of Pennsylvania.

Media, Reagents, Abs, and Flow Cytometry

All cells were grown in RPMI 1640 (Mediatech Cellgro, Herndon, Va.) supplemented with 10% heat-inactivated FCS, 100 U/ml penicillin, 100 μg/ml streptomycin, 2 mM L-glutamine, 10 mM HEPES (all from Life Technologies, Rockville, Md.), 50 μM 2-mercaptoethanol (Sigma-Aldrich, St. Louis, Mo.). FITC and biotin-anti-CD25 (7D4), FITC and allophycocyanin-anti-CD4 (RM4-5), PE-anti-CD122 (TM-β1), PE-anti-CD132 (4G3), Streptavidin-PE, purified anti-B220 (RA3), anti-MHC class II (M/5114), and purified anti-CD3 (2C11) were purchased from BD PharMingen (San Diego, Calif.). Murine rIL-2 was purchased from R&D Systems (Minneapolis, Minn.). Cells were analyzed on a Becton Dickinson FACSCaliber (BD Biosciences, Mountain View, Calif.) using FloJo software (Tree Star, San Carlos, Calif.). Anti-STAT5, anti-phospho-STAT5, anti-Akt, anti-phospho-Akt, anti-p70^{s6kinase}, anti-phospho-p70^{s6kinase}, anti-PTEN, and anti-actin Abs (all from Cell Signaling Technology, Beverly, Mass.), and anti-bcl-x_{L} (Santa Cruz Biotechnology, Santa Cruz, Calif.) were used for Western blot analysis. Anti-Foxp3 staining kit was purchased from eBioscience (San Diego, Calif.).

Cell Purification

Lymph node and spleen cells were initially prepared by lysing erythrocytes with ACK lysis buffer (Bio Whittaker, Walkersville, Md.). Cell preparations were then enriched for T cells by negative selection. Single cell suspensions were incubated with purified anti-B220 (RA3) and anti-MHC class II (M/5114) (BD Pharmingen) at 1 μg/ml for 30 min at 4° C. followed by incubation with microbead-conjugated goat anti-rat IgG (Polysciences, Warrington, Pa.) for 30 min at 4° C. Cell preps were placed on a magnetic stand for 10 minutes and the negative fraction was harvested. Single cell suspensions of about 3×10⁸ were subsequently labeled with biotin-anti-CD25 (7D4), allophycocyanin-anti-CD4 (RM4-5), and Streptavidin-PE and purified by flow cytometry on a FACSVantage Cell Sorter (BD Biosciences). The percentage of CD4⁺CD25⁺ T cells was typically 4-6% of the overall T cell pool and the purity of sorted CD4⁺CD25⁺ T cells was consistently >95%. These cells functioned as Tregs when assessed for their ability to suppress CD4⁺CD25⁻T cell proliferation in vitro coculture assays.

CD4+CD25+ T Cell Isolation

Spleen and lymph node cells were isolated from 2-3 week old Pten^{flox/flox}Cre+ mice before the onset of disease and from age-matched Cre negative littermates. Cell preparations were stained with anti-CD4APC, anti-CD25 biotin, streptavidin-PE and anti-CD45RB-FITC and subsequently purified into CD4+CD25+CD45RB^{lo} cells by flow cytometry on a FACSVantage Cell Sorter (BD Biosciences). Cell purity was routinely greater than 95%.

Bone Marrow Chimeras:

C57BL/6 Thy1.1 mice were lethally irradiated (1000 rads) prior to reconstitution with 2×10⁶ total T-depleted bone marrow cells at specified ratios from either wild-type (Thy1.1) or PTEN-ΔT (Thy1.2) mice. Recipient mice were treated with Neosporin (DSM Pharm. Inc., Greenville, NC) for one week prior to and two weeks after reconstitution. Ten weeks after reconstitution thymic subsets were analyzed by flow cytometry for expression of Foxp3 and degree of chimerism.

Survival Assays

FACS-sorted CD4⁺CD25⁺ or CD4⁺CD25⁻ cells (1×10⁶ cells/ml) were cultured in complete medium in 48-well plates (Costar, Corning, Corning, N.Y.) with or without 100 U/ml murine rIL-2 (R&D Systems) for 96 h. Absolute cell numbers were determined by counting cells on a hemocytometer using trypan blue (Sigma-Aldrich) exclusion. In addition, cells were stained with the vital dye 7-amino actinomycin D (7-AAD; Calbiochem, La Jolla, Calif.) and cell viability was assessed by flow cytometry.

CD3 and CD28 Stimulation

Latex beads (Interfacial Dynamics, Portland, Oreg.) were coated with either anti-CD3 (2 μg/ml) and/or anti-CD28 (10 μg/ml) as previously described (Ermann et al., 2001, J. Immunol. 167:4271), and resuspended at 3×10⁶ beads/ml. FACS sorted CD4⁺CD25⁺ or CD4⁺CD25⁻ cells (1×10⁶ cells/ml) were rested overnight in complete medium at 37° C. of 5% CO₂ and then stimulated with anti-CD3 (10 μg/ml) and/or anti-CD28 (10 μg/ml) coated beads for the indicated times at a 3:1 ratio of beads to T cells. Cells were subsequently lysed and analyzed by Western blotting.

IL-2 Stimulation and T Cell Priming Plus IL-2 Stimulation

FACS sorted CD4⁺CD25⁺ or CD4⁺CD25⁻ cells (1×10⁶ cells/ml) were rested overnight in complete medium at 37° C. of 5% CO₂ and then stimulated with 100 U/ml IL-2 (R&D Systems) for the time indicated, or cultured in 96-well plates (Costar, Corning) with T-depleted irradiated (2500 rads) BALB/c APCs (2×10⁶ cells/ml) and 1 μg/ml anti-CD3 for 72 h at 37° C. of 5% CO₂. Cells were subsequently washed and rested overnight in complete medium, then stimulated with 100 U/ml IL-2 as disclosed elsewhere herein. Cells were then lysed and analyzed by Western blotting. “Primed” CD4⁺ T cells referred to in this study indicate CD4⁺CD25⁻ T cells activated with anti-CD3, with or without anti-CD28 as indicated, to induce expression of the high-affinity IL-2R.

Western Blotting Analysis

Cells were lysed at 4° C. at a concentration of 1×10⁴ cells/μl. The lysis buffer was composed of 50 mM Tris-HCL (pH 6.8), 0.2% 2-mercaptoethanol, 20% glycerol, 4% SDS, and 0.001% bromphenol blue (all from Sigma-Aldrich). Cell lysates were clarified by centrifugation at 11,000×g for 10 min. Supernatants were boiled for 10 min, separated on a 10% SDS-PAGE gel at about 1×10⁶ cell equivalents/well, and blotted onto Hybond ECL nitrocellulose membranes (Amersham Pharmacia Biotech, Buckinghamshire, U.K.). The membranes were blocked overnight in blocking reagent (Boehringer Mannheim, Mannheim, Germany) at 4° C. and probed with indicated Abs at 1/1000 dilution overnight at 4° C. Membranes were washed and probed with a HRP-conjugated anti-rabbit or anti-mouse Ab at 1:1000 for 60 minutes at room temperature. Blots were visualized by ECL (Roche Diagnostics, Indianapolis, Ind.) according to the manufacturer's protocol and on Hyperfilm ECL (Amersham Pharmacia Biotech). Abs were subsequently stripped off membranes for reprobing using Restore Western Blot Stripping buffer (Pierce, Rockford, Ill.), and reprobed as disclosed elsewhere herein.

Immunoblotting:

As a result of the necessary breeding strategies employed, the numbers of PTEN-ΔT mice generated per litter were typically 50% the number of littermate controls. Therefore in order to obtain sufficient cell numbers to analyze intracellular signaling pathways, it was necessary to expand isolated PTEN-ΔT Tregs in vitro in the presence of rIL-2 as described above, while Tregs from littermate control mice could be generated in numbers allowing analysis immediately subsequent to isolation. Both cell types were subsequently rested overnight in complete medium before study. After stimulation, cells were lysed at 4° C. in lysis buffer composed of 50 mM Tris-HCL (pH 6.8), 0.2% 2-mercaptoethanol, 20% glycerol, 4% SDS, and 0.001% bromophenol blue (all from Sigma Chemical, St. Louis Mo.). Cell lysates were clarified by centrifugation at 11,000 g for 10 min. Supernatants were boiled for 5 min, separated on a 10% SDS-polyacrylamide gel (SDS-PAGE) at 1×10⁶ cell equivalents/well and blotted onto Hybond ECL nitrocellulose membranes (Amersham Pharmacia Biotech, Buckinghamshire, England). Membranes were blocked for one hour in blocking reagent (Boehringer Mannheim, Germany) at room temperature and probed with indicated antibodies at 1:1000 dilution overnight at 4° C. Membranes were washed and probed with a horseradish peroxidase-conjugated anti-rabbit or anti-mouse antibody at 1:1000 for 60 min at room temperature. Blots were visualized by enhanced chemiluminescence (Roche Diagnostics Corporation, Indiana) according to the manufacturer's protocol and on Hyperfilm ECL (Amersham Pharmacia Biotech, Buckinghamshire, England). Antibodies were subsequently stripped from membranes using Restore™ Western Blot Stripping Buffer (Pierce, Rockford, Ill.), and reprobed as above.

Retroviral Transduction:

The cDNA for human Pten, a kind gift from K. Yamada (NIH), was cloned into the MIG-IRES truncated nerve growth factor receptor retroviral vector, which has been previously described. High titer retroviral supernatants were prepared as previously described. PTEN-ΔT Tregs or primary CD4+ T cells were CFSE labeled and stimulated for 18 hours with PMA (0.2 μg/ml) and ionomycin (1 μg/ml) for 18 hours. Cells were washed and transduced by spin infection with retroviral supernatants containing 2 μg/ml polybrene (Sigma-Aldrich, St. Louis, Mo.) at 2500 rpm for 90 mins. Cells were subsequently cultured for 48-72 hours in complete media supplemented with rIL-2. Cells were analyzed for transduction efficiency by measuring NGFR expression by flow cytometry.

Quantitative Real-Time PCR:

Total RNA was extracted from FACS purified CD4+CD25+ and CD4+CD25− T cells with an RNAeasy kit (Qiagen, Valencia, Calif.) and reverse transcription performed with the Superscript first strand synthesis system (Invitrogen, Carlsbad, Calif.). Quantitative real time PCR was performed with PRISM 7700 (PE Applied Biosystems, Foster City, Calif.) using primers, with an internal fluorescent probe, specific for FoxP3 and GAPDH obtained from PE Applied Biosystems.

In Vivo BrdU Labeling:

Mice were injected i.p. with 1 mg BrdU (Pharmingen) every 12 hours for 3 days. Mice were then sacrificed and 2×10⁶ splenocytes or thymocytes were surface stained as above for CD4, CD8 and CD25. BrdU staining with anti-BrdU FITC was performed using a BrdU labeling kit (Pharmingen) as per the manufacturers instructions.

Fluorescence-Linked Immunosorbent Assay:

10⁷ 5 μm latex beads (interfacial dynamics) were coated with anti-IL-2 capture Ab (10 μg/ml) (Pharmingen) in PBS for 90 mins at 37° C. 2×10⁵ beads were added to 100 μl of test supernatant or titrated amounts of rIL-2 in 100 μl complete medium as standards. Bead bound IL-2 was detected using PE-labeled anti-IL-2 (Pharmingen) and subsequent FACS analysis.

PI3K Assay

CD4⁺CD25⁺ T cells were isolated by flow cytometry as discussed elsewhere herein. For control purposes, BALB/c total splenocytes were stimulated for 48 hours with soluble anti-CD3 before washing and rested in serum-free medium for 4 hours. Cells (6×10⁶/sample) were stimulated with rIL-2 (R&D Systems) for the indicated time points and subsequently lysed in RIPA buffer at 4° C. for 30 minutes. Debris was separated by centrifugation at 12,000×g for 15 min at 4° C. To immunoprecipitate PI3K, lysates were incubated overnight at 4° C. with 2.5 μg anti-p85 (Upstate Biotechnology, Lake Placid, N.Y.) followed by incubation at 4° C. for 4 hours with 25 μl of protein A-agarose beads (Merida et al., 1993, J. Biol. Chem. 268:6765; Karnitz et al., 1995, Mol. Cell. Biol. 15:3049). Precipitated protein pellets were washed twice with ice-cold RIPA lysis buffer and twice with ice-cold kinase buffer before resuspension in 20 μl of kinase buffer (40 mM HEPES, pH 7.5, 2 mM EGTA, 6 mM MgCl₂, 1 mM DTT, 2.5 mM PMSF, 5 mM NaCl, 0.2 mM EDTA, and 10 μM unlabelled ATP). Lipid substrate (phosphatidylinositol; Matreya, State College, Pa.) was freshly prepared by the addition of 1% v/w cholic acid and sonication for 5 minutes on ice. A total of 10 μg of lipid substrate was added to each sample followed by incubation at 25° C. for 10 minutes. This was followed by the addition of 20 μCi (0.74 MBq) of [γ-³²P]ATP per sample and a further incubation of 20 minutes at 25° C. The reaction was stopped by the addition of 5 N HCl (20 μl) and was then extracted with 160-μl mixture of chloroform-methanol (60:100) and nonaqueous fraction was separated by thin layer chromatography and developed with CHCl₃/MeOH/NH₄OH/H₂O (129:114:15:21). TLC plates were dried at room temperature and visualized by autoradiography.

DNA Microarray Hybridization and Analysis

CD4⁺CD25⁺ T cells (3×10⁶) were cultured in the presence or absence of rIL-2 (100 U/ml) for 12 hours. Alternatively CD4⁺CD25⁻T cells were stimulated with plate bound anti-CD3 (10 μg/ml) for 24 hours to induce functional IL-2R expression and washed extensively before culture in the presence or absence of rIL-2 (100 U/ml) for a further 12 hour. After stimulation, RNA was purified using RNeasy columns (Qiagen, Valencia, Calif.) according to manufacturers instructions. Total RNA was quantitatively amplified using two cycles of standard cDNA synthesis and in vitro transcription as described in Eberwine et al. (1992, Proc. Natl. Acad. Sci. USA 89:3010). In the second cycle of IVT synthesis, biotin-ribonucleotides were incorporated to produce labeled antisense RNA (Enzo Bioarray; Enzo Diagnostics, Farmingdale, N.Y.). Labeled cRNA was fragmented at 94° C. for 35 minutes in fragmentation buffer (40 mM Tris acetate, pH 8.1, 30 mM MgOAc, 100 mM KOAc) and subsequently hybridized to MG U74Av2 chips (Affymetrix, Santa Clara, Calif.) and stained with Streptavidin-PE as per manufacturers instructions. Analysis of DNA microarray data was conducted using Microarray Suite 5.0 software using manufacturer recommended parameters (Affymetrix). Two independent experiments were conducted for each stimulation condition. For alterations in expression to be classified as “real,” a specific gene probe was required to be called as increased or decreased with a signal log ratio of at least one in two independent experiments. Fold increase or decrease in expression as shown in Table I is calculated from signal log ratio values.

TABLE I
Genes with altered expression in both
CD4⁺CD25⁺ T cells and primed CD4⁺
T cells in response to IL-2 stimulation^{a}

| | | Average Fold Change^{b} | |
|---|---|---|---|
| | Accession | | Primed |
| Gene Name | No. | CD4⁺CD25⁺ | CD4⁺ |
| c-Myc | L00039 | 11.3 | 2.3 |
| IL-2R α-chain (CD25) | M26271 | 9.8 | 7.0 |
| TNF-β | M16819 | 8.6 | 14.9 |
| SOCS-2 | U88327 | 8.0 | 4.0 |
| EST | AW046694 | 8.0 | 3.7 |
| Smfn (small fragment | AI839882 | 4.9 | 3.5 |
| nuclease) | | | |
| Cytokine-inducible | D89613 | 4.6 | 3.3 |
| SH2-containing protein | | | |
| SOCS-1 | U88325 | 4.3 | 5.3 |
| Bcl-2 | L31532 | 4.0 | 2.3 |
| Xbp-1 | AW123880 | 3.5 | 7.5 |
| Reticulocalbin | D13003 | 2.9 | 3.3 |
| Agpat3 (1-acylglycerol- | AW124201 | 2.1 | 2.5 |
| 3-phosphate- | | | |
| O-acyltransferase 3) | | | |
| Zinc finger protein | AV371846 | −2.5 | −3.0 |
| IL-7R | M29697 | −2.8 | −6.1 |
| α-Actinin | J04181 | −3.0 | −3.0 |

^{a}Genes previously characterized as being induced by STAT5-mediated signaling are italicized.
^{b}Mean fold alteration in expression for each gene is calculated from two independent experiments for both CD4⁺CD25⁺ T cells and primed CD4⁺ T cells.

Proliferation Assays and Cell Cycle Analysis

FACS-purified CD4⁺CD25⁻ and CD4⁺CD25⁺ cells were cultured either in the presence of rIL-2 (100 U/ml) or plate bound anti-CD3 with or without anti-CD28 for 48 hours. After 48 hours cells were washed and replated either in the presence or absence of rIL-2 (20 U/ml) for a further 48 hours. Cultures were pulsed with [³H]thymidine for the final 16 hours and harvested. All proliferation experiments were set up in triplicate and results are expressed as a mean of triplicates. For cell cycle analysis, 1×10⁵ freshly isolated or cultured CD4⁺CD25⁺ T cells were fixed in 1 ml of 70% cold ethanol, and stored at −20° C. overnight. Samples were centrifuged at 250×g and supernatants were aspirated. Cell pellets were resuspended with 400 μL of a solution containing: 0.5 ml of propidium iodide (20 μg/ml, Sigma-Aldrich), 1000 U of RNase A (10 mg/ml, heat-inactivated in TE buffer, Sigma-Aldrich), and 10 ml of buffer (1 g/ml glucose in 1×PBS). Cells were stained for 30 minutes at room temp and subsequently analyzed on a FACSCalibur for DNA content.

In Vitro Suppression Assays:

1.5×10⁵ Thy1.1+CD4+CD25−CD45RB^{hi} T cells were CFSE labeled and cultured with 3×10⁵ irradiated T depleted splenocytes and anti-CD3 Ab (2C11; 0.5 μg/ml). The indicated ratios of CD4+CD25+CD45RB^{lo} cells from either wild type or PTEN-ΔT mice (Thy1.2+) were added to the cultures. To measure suppression, CFSE dilution of Thy1.1 positive cells was assessed by flow cytometry after 72 hours. Alternatively, cells were pulsed with 0.5 uCi of tritiated thymidine for the final 12 hours before being harvested.

In Vivo Colitis Model:

Rag1^{-/-} mice were injected i.v. with 6×10⁵ CD4+CD25−CD45RB^{hi} T cells either alone or with 3×10⁵ freshly isolated wild type CD4+CD25+CD45RB^{lo} Tregs or PTEN-ΔT Tregs which had been expanded in vitro with rIL-2 (100 U/ml). Mice were weighed and examined every week for signs of disease and sacrificed for tissue harvest at 8 weeks. Tissues were fixed in 10% neutral buffered Formalin (Fisher, Swedesboro, N.J.), cut into 5 μm sections and stained with H&E. Severity of colitis was scored blindly as described previously

IL-2 is a Survival Factor for CD4⁺CD25⁺ Tregs

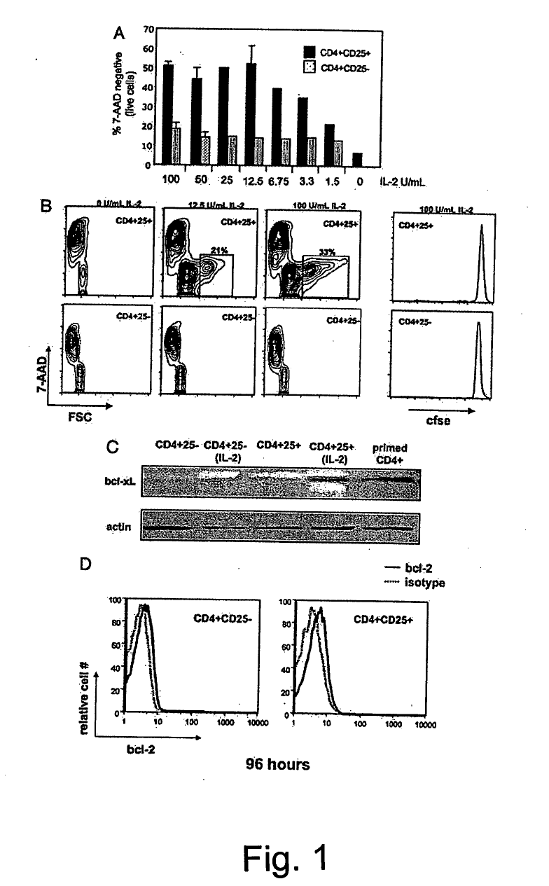

It has been demonstrated that IL-2 signals are required for the maintenance of CD4⁺CD25⁺ T cells in vivo (Malek et al., 2002, Immunity 17:167; Murakami et al., 2002, Proc. Natl. Acad. Sci. USA 99:8832). To directly determine whether exogenous IL-2 could protect Tregs from apoptosis in vitro, FACS purified CD4⁺CD25⁺ or CD4⁺CD25⁻T cells were cultured in medium for 96 hours with or without exogenous IL-2, subsequently stained with the vital dye 7-AAD, and examined by flow cytometry. Although the addition of IL-2 to CD4⁺CD25⁻T cells in culture had little effect on their viability, titration of exogenous IL-2 into cultures demonstrated that <3 U/ml IL-2 markedly increased the survival of CD4⁺CD25⁺T cells (FIG. 1A). Similarly, absolute cell counts determined by trypan blue exclusion on a hemocytometer demonstrated that the addition of IL-2 to CD4⁺CD25⁺ T cells in culture over a 96-hour period resulted in an about 5-fold increase in the number of cells recovered when compared with cells cultured in medium alone. CD4⁺CD25⁺ T cells cultured in the presence of exogenous IL-2 exhibited marked cellular enlargement (FIG. 1B) and enhanced cell survival occurred without any appreciable cell division as assessed by CFSE dilution (FIG. 1B, right panel). These observations are consistent with the known inability of IL-2 alone to induce the proliferation of highly purified CD4⁺CD25⁺ T cells (Thornton et al., 1998, J. Exp. Med. 188:287). The ability of IL-2 to promote the survival of CD4⁺CD25⁺ Tregs was observed to be associated with induction of bcl-x_{L} protein (FIG. 1C), whereas the effects on bcl-2 levels were far more modest, with only a minimal and variable increase seen (FIG. 1D).

Distinct IL-2R Signaling in CD4⁺CD25⁺ Tregs

The high-affinity IL-2R is a heterotrimeric complex composed of the α-chain (CD25), β-chain (CD122), and common γ-chain (CD132). Binding of IL-2 to the IL-2R initiates a complex signaling cascade that can drive the proliferation of Ag-activated T cells. Although CD4⁺CD25⁺ Tregs appear to constitutively express all three subunits of the IL-2R (Malek et al., 2002, Immunity 17:167; Levings et al., 2001, J. Exp. Med. 193:1295), studies on CD4⁺CD25⁺ T cells have clearly demonstrated that these cells are hypoproliferative to IL-2 in vitro (Thornton et al., 1998, J. Exp. Med. 188:287). To better understand the mechanism(s) by which CD4⁺CD25⁺ T cells remain hypoproliferative to IL-2 signals, experiments were designed to characterize signaling events downstream of the IL-2R.

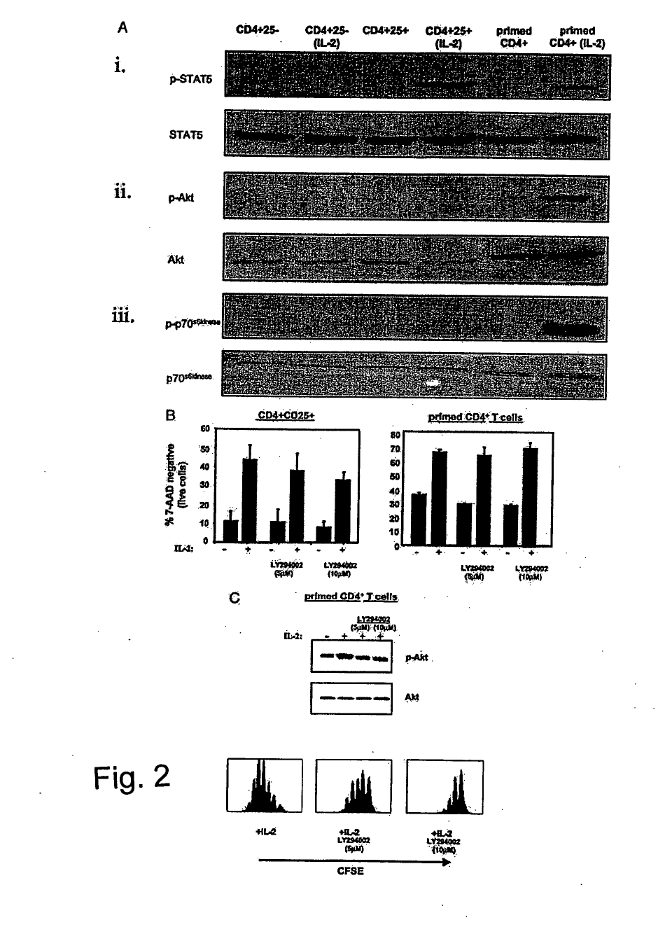

Previous studies on IL-2R signaling have shown that activation of both the JAK/STAT and PI3K/Akt pathways are critical for IL-2-induced proliferation (Brennan et al., 1997, Immunity 7:679; Moriggl et al., 1999, Immunity 10:249; Ahmed et al., 1997, Proc. Natl. Acad. Sci. USA 94:3627; Reif et al., 1997, Biol. Chem. 272:14426; Van Parijs et al., 1999, Immunity 11:281). Thus, the activation state of STAT5 and Akt downstream of the IL-2R in CD4⁺CD25⁺ T cells were examined. To this end, purified CD4⁺CD25⁺ T cells or CD4⁺CD25⁻ T cells, were rested overnight in medium and subsequently stimulated with 100 U/ml IL-2 for 30 minutes. In addition, to serve as a positive control for IL-2R signaling in this and future experiments, CD4⁺ T cells (designated “primed CD4+” in the study) were activated with anti-CD3 for a period of 72 hours to induce expression of the high-affinity IL-2R, rested overnight, and then stimulated with IL-2 as indicated. Similar to primed CD4⁺ T cells, expression of phosphorylated STAT5 in response to IL-2 in CD4⁺CD25⁺ T cells was observed (FIG. 2A). Primed CD4⁺ T cells also phosphorylated Akt following incubation with IL-2. In contrast, detection of phosphorylated Akt in CD4⁺CD25⁺ T cells stimulated with IL-2 for 2, 10, or 30 minutes was not observed (FIG. 2A).

To determine whether the failure to activate Akt by IL-2R stimulation was restricted to Akt or was indicative of a broader defect in PI3K signaling, the activation of another downstream target of PI3K, the cell cycle regulator p70^{s6kinase} was examined (Reif et al., 1997, Biol. Chem. 272:14426). Similar to the observed differential pattern of Akt phosphorylation, it was observed that phosphorylation of p70^{s6kinase} in response to IL-2 occurred in primed CD4⁺ T cells, but not in CD4⁺CD25⁺ T cells (FIG. 2A). Taken together, these data demonstrate that IL-2R signaling in CD4⁺CD25⁺ Tregs is distinct from activated or primed CD4⁺ T cells.

Previous studies on activated T cells have shown that PI3K signaling and activation of Akt are critical for the antiapoptotic effects of IL-2 (Kelly et al., 2002, J. Immunol. 168:597). The ability of IL-2 to increase the viability of CD4⁺CD25⁺ T cells (FIG. 1, A and B, left panel) without the apparent activation of Akt (FIG. 2A) suggested that this effect did not require the activation of downstream PI3K targets. To confirm that the IL-2-mediated survival of Tregs was occurring in a PI3K-independent manner, CD4⁺CD25⁺ T cells were cultured with 100 U/ml IL-2 in the presence of increasing doses of the PI3K chemical inhibitor LY294002 (Gonzalez-Garcia et al., 1997, J. Biol. Chem. 272:10220) for 96 hours. At concentrations sufficient to inhibit IL-2-mediated activation of Akt as well as proliferation of primed CD4⁺ T cells (FIG. 2C), it was observed that LY294002 did not inhibit IL-2-mediated survival of Tregs or primed CD4⁺ T cells (FIG. 2B).

Distinct Transcriptional Activity of IL-2-Stimulated CD4⁺CD25⁺ Tregs

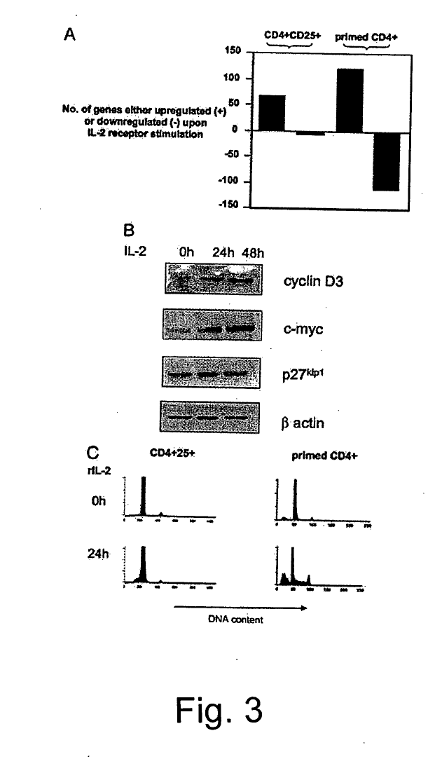

The results presented herein demonstrate a distinct pattern of IL-2R signaling in Tregs. To broadly determine whether IL-2R signaling in CD4⁺CD25⁺ T cells was capable of initiating transcriptional activity, the gene expression profile of IL-2-stimulated primed CD4⁺ T cells was compared with CD4⁺CD25⁺ T cells. Using cDNA microarray analysis it was observed that IL-2R signaling altered transcription of significantly fewer genes in CD4⁺CD25⁺ cells when compared with primed CD4⁺ cells (71 vs. 229) (FIG. 3A). This included both a reduction in the number of genes up-regulated (67 vs. 120), and more strikingly, a large disparity in the number of genes down-regulated (4 vs. 109). Further analysis of the gene array data revealed that the expression of 15 gene transcripts was altered both in CD4⁺CD25⁺ T cells and in primed CD4⁺ T cells after stimulation with IL-2 (see Table I). Significantly, of these 15 genes almost one-half (IL-2Rα, suppressor of cytokine signaling (SOCS)-1 and SOCS-2, cytokine-inducible SH2-containing protein, c-Myc, bcl-2, and TNF-β) have been previously characterized to be downstream of JAK/STAT-mediated signaling pathways (Lord et al., 2000, J. Immunol. 164:2533; Sadowski et al., 2001, J. Biol. Chem. 276:20703; Kim et al., 2001, Immunity 15:159; Lu et al., 1998, Eur. J. Immunol. 28:805; Sporri et al., 2001, Blood 97:221; Matsumoto et al., 1997, Blood 89:3148). These data demonstrate that IL-2R signaling in CD4⁺CD25⁺ T cells, although differing markedly from that observed in primed CD4⁺ T cells, is sufficient to initiate transcription and results in the specific up-regulation of several known STAT5-dependent genes.

STAT5-mediated up-regulation of cyclin D3 and c-myc and PI3K dependent down-regulation of p27^{kip} have been reported to play a critical role in IL-2-mediated cell cycle progression (Brennan et al., 1997, Immunity 7:679; Moriggl et al., 1999, Immunity 10:249; Wells et al., 2001, J. Clin. Invest. 108:895). Movement of cells out of G₀ into G₁ is characterized by the up-regulation of the G₁ cyclins whereas movement out of G₁ requires the down-regulation/degradation of the cell cycle inhibitor p27^{kip} (Gillett et al., 1998, Mol. Pathol. 51:310). Analysis of these cell cycle-associated proteins demonstrated that stimulation of Tregs with IL-2 led to the up-regulation of cyclin D3 and c-myc, but failed to down-regulate p27^{kip} (FIG. 3B). Propidium iodide staining for DNA content confirmed that IL-2 stimulation induced Tregs to arrest in G₁ phase of the cell cycle (FIG. 3C). These observations are consistent with the microarray data disclosed herein, as well as being in agreement with the observations on the failure of IL-2 to activate signaling pathways downstream of PI3K in Tregs (FIG. 2A).

Uncoupling of IL-2R Signaling in Tregs Occurs Downstream of PI3K

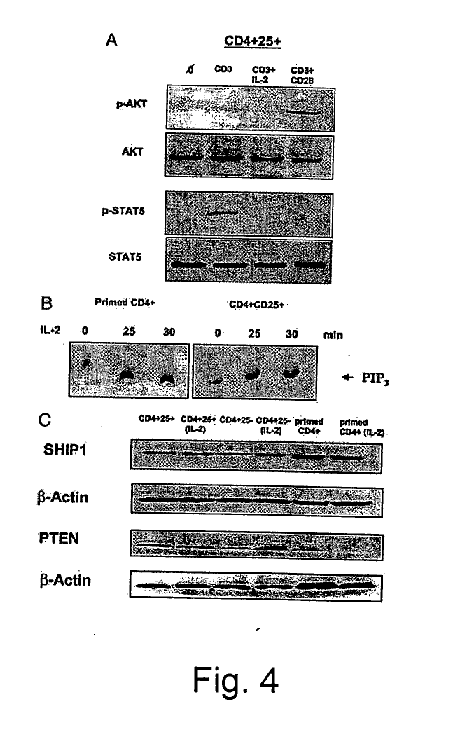

The results presented herein indicate that IL-2 stimulation of CD4⁺CD25⁺ T cells does not activate downstream targets of the PI3K signaling pathway. Without wishing to be bound by any particular theory, possible explanations for this observation include 1) CD4⁺CD25⁺ T cells are incapable of activating PI3K regardless of the stimulus, 2) a failure to activate PI3K specifically downstream of the IL-2R, or 3) downstream mediators of PI3K signaling, such as phosphatidylinositol 3,4,5-trisphosphate (PIP₃), are negatively regulated in Tregs. To address these possibilities, experiments were designed to assess whether Akt could be activated in response to other stimuli, such as TCR signals or costimulation. FACS sorted CD4⁺CD25⁺ T cells were rested overnight in culture medium and stimulated with anti-CD3, anti-CD3 plus IL-2 or anti-CD3 plus anti-CD28 for 30 minutes. Cells were subsequently lysed and probed for phosphorylated Akt or phosphorylated STAT5 by Western blot. TCR signals alone or in conjunction with IL-2 did not activate Akt in Tregs (FIG. 4A). However, phosphorylated Akt in response to anti-CD3 plus anti-CD28 or anti-CD28 alone was not observed (FIG. 4A), indicating that PI3K can be activated in CD4⁺CD25⁺ T cells. STAT5 phosphorylation confirmed the observation that IL-2 is capable of STAT5 activation in CD4⁺CD25⁺ T cells. Thus, CD28 cross-linking is capable of activating the PI3K signaling pathway, ruling out an intrinsic defect in PI3K activity in Tregs.

Activation of PI3K by CD28 and the IL-2R differ in that the IL-2R does not have intrinsic docking sites for PI3K (Fruman et al., 2002, Semin. Immunol. 14:7). Activation of PI3K by IL-2 relies on the assembly of a scaffolding complex composed of proteins, such as SHC and Grb2, which leads to the subsequent recruitment of PI3K to the IL-2R (Gu et al., 2000, Mol. Cell. Biol. 20:7109). Once activated, PI3K signaling is negatively regulated by the lipid phosphatases PTEN and Src homology 2-containing inositol polyphosphate 5-phosphatase (SHIP)-1, which convert active lipid messengers to less active intermediates (Freebum et al., 2002, J. Immunol. 169:5441). Therefore, the failure to activate Akt downstream of the IL-2R in CD4⁺CD25⁺ T cells could result from either the inability to assemble this proximal PI3K signaling complex or the negative regulation of PI3K signaling intermediates. To distinguish between these possibilities, an in vitro PI3K assay was used. In this assay, the activation of PI3K by IL-2 is measured by determining IL-2-mediated phosphorylation of PI3K substrates in vitro. As indicated in FIG. 4B, stimulation of CD4⁺CD25⁺ T cells with IL-2 led to the induction of specific PI3K activity, similar to that observed in IL-2-stimulated activated CD4⁺ T cells (Brennan et al., 1997, Immunity 7:679). Thus, proximal IL-2R signaling in CD4⁺CD25⁺ T cells is sufficient to activate PI3K and suggests that regulation of this pathway lies downstream of PI3K.

Having determined that IL-2 activates PI3K in Tregs, the expression pattern of the lipid phosphatases PTEN and SHIP-1 in naive CD4⁺, CD4⁺CD25⁺, and primed CD4⁺ T cells was next examined. Western blot analysis revealed that both PTEN and SHIP-1 are expressed at high levels in naive CD4⁺ and CD4⁺CD25⁺ T cells, whereas primed CD4⁺ T cells had low levels of PTEN, but maintained SHIP-1 expression (FIG. 4C). The expression pattern of PTEN in CD4⁺CD25⁺ and primed CD4⁺ T cells mimics observed PI3K activity and IL-2 responsiveness, suggesting a potential mechanism for the negative regulation of the PI3K pathway distal to the IL-2R in CD4⁺CD25⁺ T cells.

TCR Priming Down-Regulates PTEN Expression and Facilitates IL-2-Induced Proliferation of CD4⁺CD25⁺ T Cells

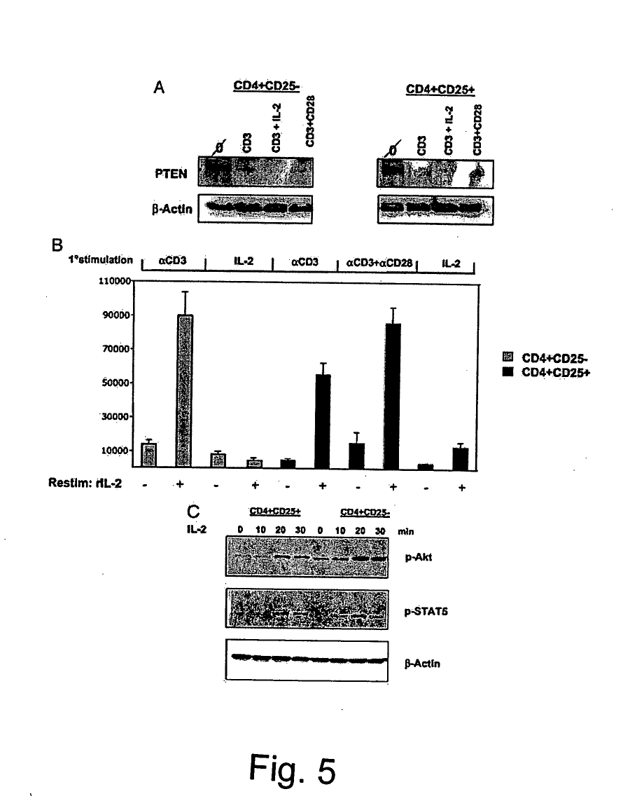

CD4⁺CD25⁺ T cells are hypoproliferative to either TCR or IL-2R stimulation alone, however both of these signals in combination restores the proliferative capacity of Tregs (Thornton et al., 1998, J. Exp. Med. 188:287), which can be subsequently maintained by IL-2 alone. It has been observed that PTEN expression levels inversely correlate with the proliferative response of primed CD4⁺ T cells to exogenous IL-2 as well as the hypoproliferative phenotype of CD4⁺CD25⁺ T cells to IL-2 signals. Therefore, experiments were designed to determine whether TCR stimulation of CD4⁺CD25⁺ T cells altered the expression of PTEN. As shown in FIG. 5A, TCR or TCR/CD28 stimulation resulted in the down-regulation of PTEN after 72 hours.

Having established that TCR stimulation results in the down-regulation of PTEN in CD4⁺CD25⁺ T cells, it was next assessed whether TCR signals can restore IL-2R competency to support IL-2-induced proliferation. To address this, CFSE labeled CD4⁺CD25⁺ T cells were cultured with IL-2 alone or plate-bound anti-CD3 with or without anti-CD28 for 48 hour. It was observed that CD4⁺CD25⁺ T cells did not undergo significant proliferation to IL-2 or anti-CD3 stimulation alone or with anti-CD28 during this period. After 48 hours, the cultured CD4⁺CD25⁺ T cells were washed and recultured in medium alone or with 20 U/ml IL-2 as indicated for a further 72 h. CD4⁺CD25⁺ T cells cultured with IL-2 underwent significant proliferation (FIG. 5B), suggesting that indeed, an initial period of T cell activation restores IL-2R-mediated proliferation. However, the level of IL-2-induced proliferation of Tregs after a period of preactivation with anti-CD3 alone is less than that observed in CD4⁺CD25⁻ T cells under the same conditions. This observation raised the possibility that other unidentified factors intrinsic to Tregs besides PTEN may also play a role in their hyporesponsiveness to IL-2.

The results here in have demonstrated that TCR activation results in the down-regulation of PTEN and restores the ability of CD4⁺CD25⁺ T cells to proliferate to IL-2. Thus, it was next assessed whether TCR stimulation restored IL-2 mediated PI3K signaling. To this end, CD4⁺CD25⁺ T cells were activated for 72 hours with anti-CD3 and anti-CD28, rested in medium for 6 hours and restimulated with IL-2 (FIG. 5C). In contrast to freshly isolated CD4⁺CD25⁺ T cells (FIG. 2A), it was observed that activated CD4⁺CD25⁺ T cells phosphorylated Akt in response to IL-2. Taken together, these data demonstrate that TCR stimulation of CD4⁺CD25⁺ T cells primes the IL-2R for IL-2-mediated proliferation, coincides with the loss of PTEN, and the restoration of PI3K signaling downstream of the IL-2R.

Distinct IL-2 Receptor Signaling Pattern in CD4⁺CD25⁺ Regulatory T Cells

In contrast to naive T cells, CD4⁺CD25⁺ immunoregulatory T cells constitutively express the α-, β-, and common γ-chain of the IL-2R (Malek et al., 2002, Immunity 17:167, Levings et al., 2001, J. Exp. Med. 193:1295). Despite expression of the high-affinity IL-2R, initial characterization of CD4⁺CD25⁺ T cells demonstrated that these cells are hypoproliferative to IL-2 in vitro (Thornton et al., 1998, J. Exp. Med. 188:287). One explanation for these data would be that Tregs do not express a “functional” IL-2R. However, subsequent studies have shown that CD4⁺CD25⁺ T cells require IL-2 and IL-2R signaling for their development and survival (Malek et al., 2002, Immunity 17:167; Murakami et al., 2002, Proc. Natl. Acad. Sci. USA 99:8832; Almeida et al., 2002, J. Immunol. 169:4850), suggesting that these cells are responsive to IL-2 signals. The mechanism(s) by which IL-2R signaling on CD4⁺CD25⁺ T cells promotes their development and survival, yet maintains their hypoproliferative phenotype is unknown. To that end, the experiments and results presented herein address the cellular and molecular responses of CD4⁺CD25⁺ T cells to IL-2 in vitro. The results presented herein demonstrate that CD4⁺CD25⁺ T cells have a distinct IL-2R signaling pattern, which may in part explain their hypoproliferative phenotype while preserving IL-2-mediated survival.

The molecular mechanism(s) that regulate cytokine or growth factor-induced cell cycle progression and survival remain incompletely understood. STAT5a and STAT5b are two closely related transcription factors that are activated by the IL-2R (Lin et al., 1997, Cytokine Growth Factor Rev. 8:313), and appear to be required for IL-2-mediated proliferation in activated T cells (Moriggl et al., 1999, Immunity 10:249). Examination of STAT5a/STAT5b^{-/-} T lymphocytes demonstrated a specific role for STAT5 in linking IL-2 signals to the cell cycle via the up-regulation of cell cycle proteins such as cyclin D2, cyclin D3, and cdk 6. However, these studies also demonstrated that assembly of the cell cycle machinery and cell cycle progression is not entirely dependent on STAT5 activation. Despite a defect in the expression of some cell cycle machinery, primed STAT5-deficient T cells are able to up-regulate the cell cycle proteins cdk2 and cdk4, as well as down-regulate p27^{kip} in response to IL-2 (Moriggl et al., 1999, Immunity 10:249). Thus, efficient cell cycle progression requires coordinated signaling from multiple signaling pathways. Similar to activated T cells, it was observed that IL-2-mediated phosphorylation of STAT5 and up-regulation of the STAT5-dependent G₁ cyclin D3 in CD4⁺CD25⁺ T cells. Additionally, it was observed that IL-2 stimulation initiates the transcription of several of STAT5-dependent genes, as well as the cell cycle associated transcription factor c-myc. Thus, JAK/STAT5 signaling pathway appears to be intact in these cells.

The results presented herein demonstrate that IL-2 stimulation of Tregs increases cellular survival, which coincides with the up-regulation of the antiapoptotic molecule bcl-x_{L}. Chemical inhibitors of PI3K and MAPK signaling did not block the IL-2-mediated survival of CD4⁺25⁺ T cells (FIG. 2B), thus, demonstrating that JAK/STAT5 signaling pathway is critical for this IL-2 effect. Such a role of STAT5 in IL-2-mediated T cell survival has not been previously established prior to the present disclosure. Examination of activated T cells expressing a mutated IL-2R, or from STAT5-deficient mice have shown that STAT5 activation is not necessary for IL-2-induced protection from apoptosis (Refaeli et al., 1998, Immunity 8:615; Moriggl et al., 1999, Immunity 10:249). Without wishing to be bound by any particular theory, it is believed that a clear caveat to these studies is that multiple signaling pathways are activated downstream of the IL-2R, and as such, STAT5-mediated survival may act in a redundant manner. In contrast, studies in cell lines expressing chimeric and/or mutated growth factor receptors demonstrated that activation of STAT5 protects cells from apoptosis via the up-regulation of bcl-2 and bcl-x_{L}, independent of cell cycle progression (Lord et al., 2000, J. Immunol. 164:2533). Likewise, further studies on STAT5-deficient mice have shown that activation of STAT5 by growth factors is required for the up-regulation of the antiapoptotic molecule bcl-x_{L} and survival of erythroid progenitors (Socolovsky et al., 1999, Cell 98:181).

The results presented herein indicate that the PI3K/Akt pathway is dispensable in cytokine-mediated survival of Tregs. In support of these observations, the results presented herein demonstrate that 1) CD4⁺CD25⁺ T cells do not activate Akt in response to IL-2, 2) the PI3K chemical inhibitor LY294002 does not block the IL-2-mediated survival of Tregs, and 3) IL-2 leads to significant up-regulation of bcl-x_{L} without significantly altering bcl-2 expression levels in these cells.

Proximal IL-2R signaling in Tregs is sufficiently intact to activate PI3K, however downstream targets such as Akt do not get phosphorylated. The results presented herein suggest that regulation of this signaling pathway occurs downstream of PI3K. The lipid phosphatase PTEN is a ubiquitously expressed protein, which has been characterized as a negative regulator of PI3K-mediated signaling, through its ability to dephosphorylate lipid substrates. PTEN (also referred to as mutated in multiple advanced cancers) is, as its name suggests, a potent tumor suppressor gene that is deleted or mutated in a variety of different forms of cancer (Seminario et al., 2003, Immunol. Rev. 192:80.). Complete inactivation of the gene in mice was found to be embryonic lethal and subsequent studies on hemizygous animals demonstrated a high incidence of spontaneous tumor development, impaired Fas-mediated apoptosis, as well as autoimmunity (Di Cristofano et al., 1999, Science 285:2122; Podsypanina et al. 1999, Proc. Natl. Acad. Sci. USA 96:1563).

The results presented herein demonstrate that the inability of IL-2 to stimulate signaling pathways downstream of PI3K coincides with relatively high expression of PTEN in Tregs. Moreover down-regulation of PTEN through TCR stimulation appears to be permissive for a restoration of IL-2-mediated PI3K-dependent signaling and proliferative capacity in Tregs. These observations are in agreement with earlier observations that a combination of TCR and IL-2 signals are sufficient to reverse the hypoproliferative phenotype of Tregs (Thornton et al., 1998, J. Exp. Med. 188:287). It is interesting that such a combination of signals does not appear to significantly activate Akt in the short term, e.g., 30 minutes (FIG. 4A) perhaps due to maintained PTEN expression at these earlier time points. Indeed, the results presented herein demonstrate that under such stimulation conditions, PTEN degradation is detectable after about 24 hours and that prolonged activation of Tregs (72 hours) facilitates IL-2-dependent signaling downstream of PI3K (FIG. 5C).

IL-2 plays a critical role in the prevention of autoimmunity and the maintenance of lymphocyte homeostasis. One such mechanism for IL-2-mediated tolerance is via the development and survival of CD4⁺CD25⁺ T cells (Malek et al., 2002, Immunity 17:167; Murakami et al., 2002, Proc. Natl. Acad. Sci. USA 99:8832; Almeida et al., 2002, J. Immunol. 169:4850). The results presented herein define a distinct pattern of IL-2R signaling in Tregs, which is regulated by PTEN and differs significantly from the established paradigm of IL-2R signaling. This signaling pattern appears to be crucial for the “anergic” phenotype of these cells to IL-2. Mice that are haploinsufficient for PTEN or have a T cell-specific PTEN deletion develop autoimmune disease (Di Cristofano et al., 1999, Science 285:2122; Suzuki et al., 2001, Immunity 14:523). In these models, it was demonstrated that effector T cells are hyperresponsive, however the Treg populations were not investigated. The results presented herein suggest that PTEN acts as a negative regulator of IL-2-mediated expansion of Tregs. Whether PTEN plays a role in their suppressive phenotype remains to be investigated. Taken together, the data presented herein describe a novel signaling paradigm that explains how IL-2 mediates the development and survival of CD4⁺CD25⁺ T cells without inducing a significant mitogenic response.

PTEN-Deficient Tregs Prevent Disease in an In Vivo Model

The model chosen in this experiment is one which is high established in the field, in which purified CD4⁺CD25⁻ T cells are adoptively transferred into T cell deficient mice (either scid or RAG-deficient mice). In this environment, the cells undergo homeostatic expansion and cause autoimmune inflammatory bowel disease. This manifests itself as failure to gain weight, and has characteristic histologic changes. In contrast, co-administration of CD4⁺CD25⁺ T cells Tregs prevented disease.

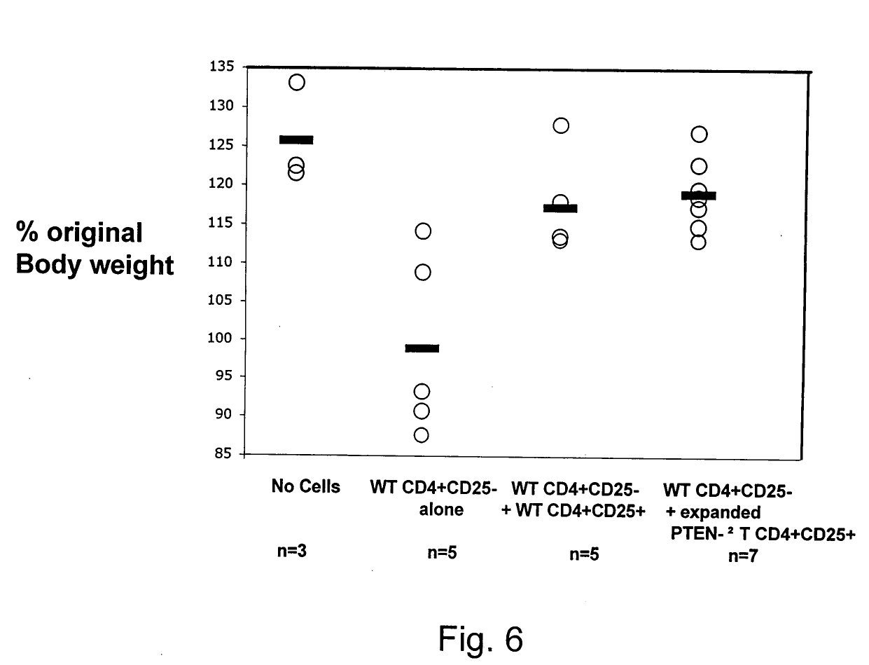

To study whether PTEN-deficient regulatory T cells had the capacity to similarly prevent disease in vivo, 3×10⁵ CD4⁺CD25⁻ B6 cells were adoptively transferred into 3 cohorts of B6 RAG-2 deficient mice. Group 1 received no additional cells, group 2 also received 600,000 B6 CD4⁺CD25⁺ cells, and group 3 also received 600,000 B6 PTEN deficient CD4⁺CD25⁺ cells (which had been cultured ex vivo for 5 days in IL-2 (FIG. 6). These data indicate that manipulation of PTEN as a means to promote regulatory cell growth does not perturb the capacity of these cells to suppress disease in vivo.

Tregs with Acquired Loss of PTEN Still Proliferate in Response to IL-2

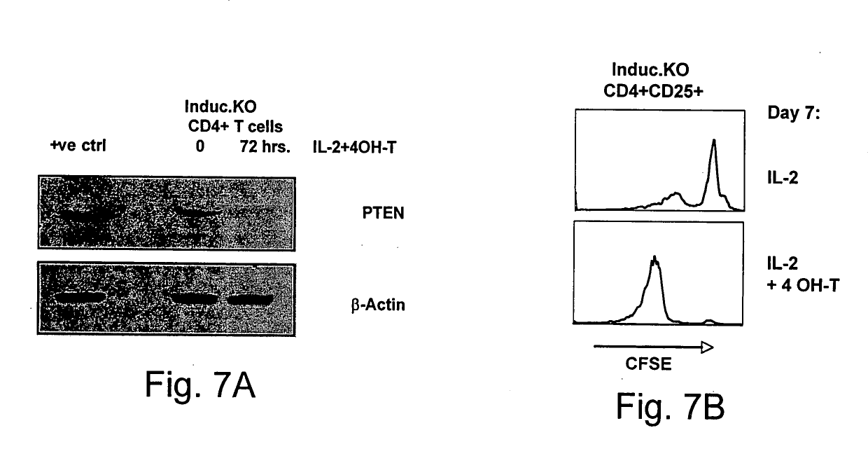

The results presented herein demonstrated that mice which genetically lacked PTEN in the T cell lineage still had CD4⁺CD25⁺ Tregs, and that these cells could be expanded ex vivo with IL-2. The experiments herein were designed to assess whether this IL-2 responsiveness would also occur in situations where otherwise normal Tregs, which developed in the context of PTEN expression, would become IL-2 responsive under conditions where PTEN loss was later acquired. To address this question, PTEN flox/flox mice were crossed with ER-Cre mice. In the top panel of FIG. 7, purified CD4⁺CD25⁻T cells were either lysed immediately or cultured with IL-2 (100 U/ml) plus 4-OH Tamoxifen (1 nM) for 72 hours. PTEN expression was assessed by Western, and the results demonstrated that by 72 hours in culture with 40H-T, PTEN expression was observed to be below the limits of detection. In the bottom panel of FIG. 7, CD4⁺CD25⁺ Tregs were labeled with CFSE and cultured with IL-2 alone or IL-2 plus 40H-T (concentrations as above). The results demonstrate that the inducible downregulation of PTEN was sufficient to confer IL-2 proliferative responsiveness on otherwise normal Tregs.

CD4+CD25+ Regulatory T Cells Develop Normally in the Absence of PTEN

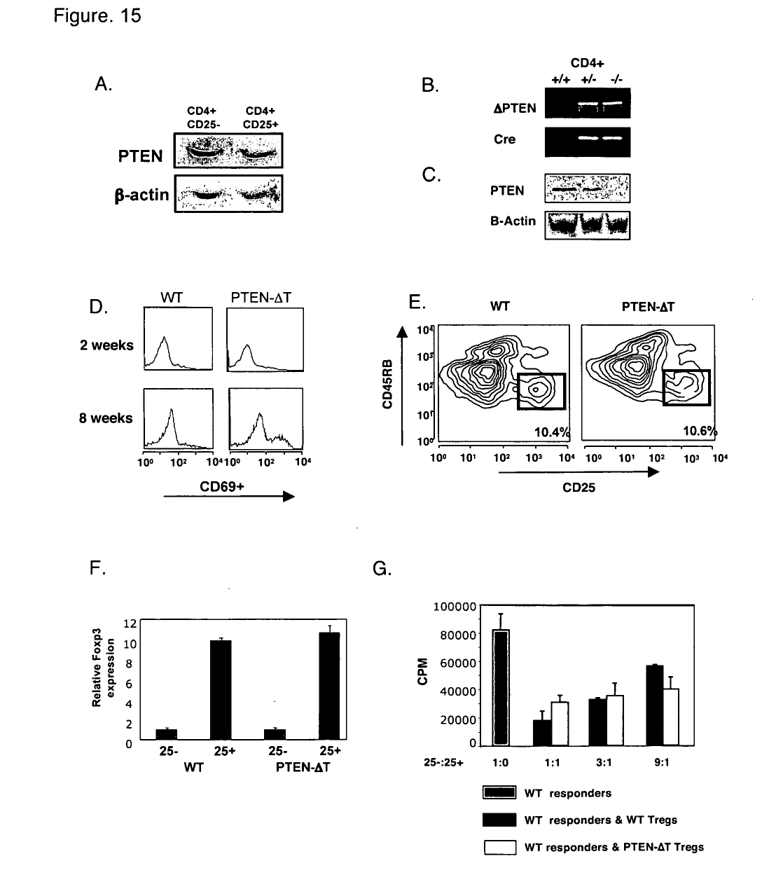

In normal mice, PTEN is expressed at equivalent levels in Treg and CD4+CD25− T cell subsets (FIG. 15A). However, as PTEN deficiency in mice results in embryonic lethality, to examine the role of PTEN in CD4+CD25+ regulatory T cells, mice were used having a targeted deletion of PTEN specific to the T cell compartment. Mice homozygous for expression of the Pten^{flox} allele were crossed with CD4-Cre transgenic mice. From the resulting litters (termed PTEN-ΔT mice), genomic DNA from purified CD4+ T cells was screened by PCR for expression of the Cre transgene. Specific recombination at the Pten^{flox} locus was detected using primers flanking the 5′ and 3′ loxP sites, which amplify a 849 bp product only after Cre mediated deletion of exons 4 and 5 (FIG. 15B). T cell specific deletion of PTEN was confirmed by western blotting for total PTEN expression in purified CD4+ T cells from 3-week old homozygous mutant Pten^{flox/flox} Cre+, heterozygous mutant Pten^{flox/+} Cre+, or wild type Cre− mice (FIG. 15C).

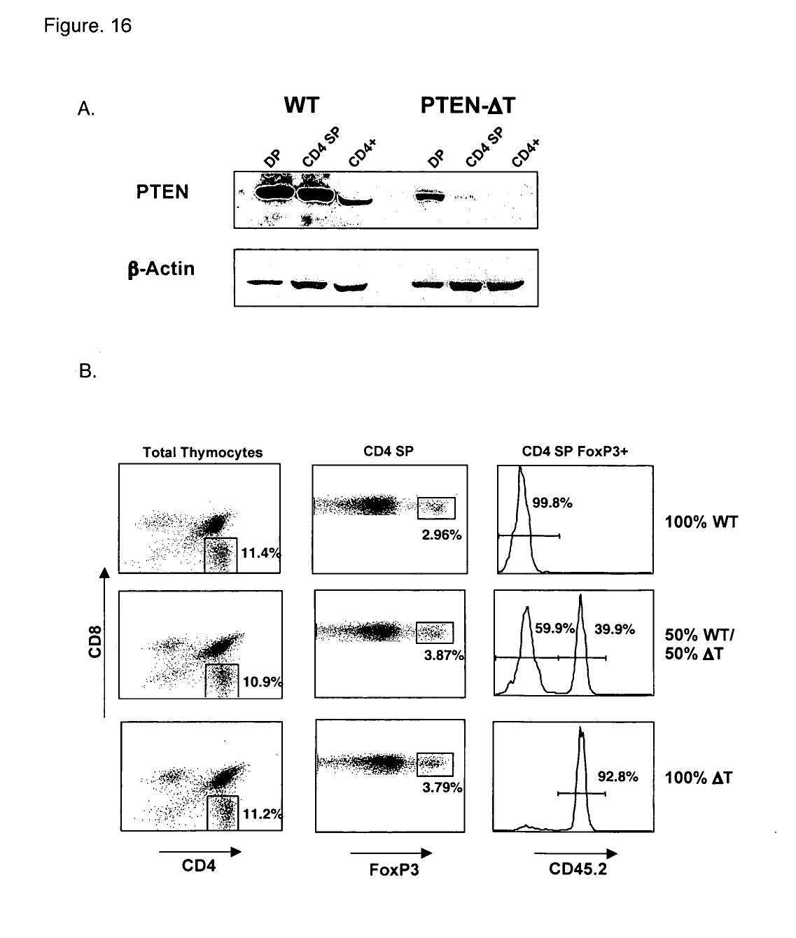

The precise phase of thymic development at which CD4+ T cells commit to the Treg lineage is controversial and although Cre recombinase becomes active during the double positive phase of T cell development in the CD4−Cre mouse, the in vivo half-life of PTEN protein expression may be sufficient that PTEN-ΔT Tregs develop in the presence of PTEN. Therefore levels of PTEN expression were examined during different phases of T cell development in the thymus of 3-week old PTEN-ΔT and wild-type mice. As shown in FIG. 16A, PTEN protein levels were notably lower (compared with littermate controls) in double positive thymocytes from PTEN-ΔT mice, and were almost undetectable in CD4+ single positive thymocytes.

To further determine whether PTEN deficiency affects Treg development bone marrow chimeric mice were generated by reconstituting lethally irradiated congenic hosts with wild type (Thy1.1) and/or PTEN-ΔT (Thy1.2) bone marrow. Mice were reconstituted with either 100% wild type, 100% PTEN-ΔT or with a 50%/50% mixture of bone marrow derived from both mice. Ten weeks after reconstitution, thymic development appeared normal in all chimeras with similar percentages of double negative, double positive and single positive subsets (FIG. 16B left panels). Furthermore examination of Foxp3 expression among CD4 single positive cells from all groups demonstrated equivalent levels of Treg development (FIG. 16B middle panels). To analyze the relative proportion of wild type and PTEN-ΔT derived cells within the CD4+ Foxp3+Treg subsets of the mixed chimeras, expression of congenic Thy1 markers was examined. As shown in FIG. 16B (right panels), although the proportion of wild-type derived Tregs is slightly higher in the 50/50 chimeric mice (59.9% vs. 39.9%), this is most likely due to residual host-derived T cell development, similar to what is seen in the 100% PTEN-ΔT chimeric mice in which 92.8% of Tregs are PTEN deficient. Together these data indicate that CD4+CD25+ regulatory T cells develop normally in the absence of PTEN.

Intact Suppression by PTEN-Deficient CD4+CD25+Regulatory T Cells

Previous reports have demonstrated that loss of PTEN in the T cell compartment, achieved by crossing Pten^{flox/-} mice with lck-Cre transgenic mice, results in lethality by about 15 weeks of age due to the development of CD4+ T cell lymphomas. Mice used in the present study had a very similar phenotype, developing CD4+ T cell lymphomas by 10-12 weeks. In agreement with these reports, lymphoma was preceded by an increased total number of CD4+ lymphocytes and an accumulation of activated CD4+ T cells in the periphery, which became apparent after 6 weeks. This accumulation of activated CD4+ T cells in the periphery was detected as a significant increase in the numbers of CD4+ cells expressing the activation marker CD69 (FIG. 1D). As previously reported, the expression levels of CD25 on CD4+ T cells did not vary significantly between PTEN-ΔT mice and wild type littermate controls.

To avoid contamination of putative regulatory T cells with recently activated T cells, mice were sacrificed at 2-3 weeks of age, a time at which no increase in the number of CD69+CD4+ T cells was detectable (FIG. 15D). Phenotypic analysis of PTEN-ΔT mice demonstrated similar numbers of CD4+CD25+CD45RB^{lo} cells when compared to littermate controls (FIG. 15E). After purification by FACS, real time PCR demonstrated that cells from both PTEN-ΔT and control mice expressed comparable levels of Foxp3 mRNA (FIG. 15F). Furthermore, PTEN-ΔT Tregs were able to suppress the proliferation of wild type responder cells in vitro to the same extent as Tregs isolated from control mice (FIG. 15G). Taken together these data demonstrate that CD4+CD25+ regulatory T cells develop normally in the absence of PTEN.

PTEN Deficient CD4+CD25+ Regulatory T Cells Proliferate in Response to IL-2

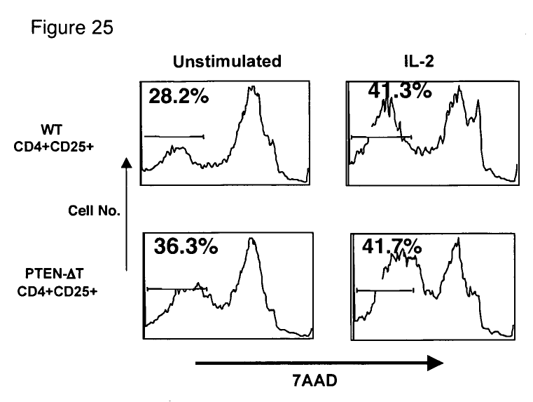

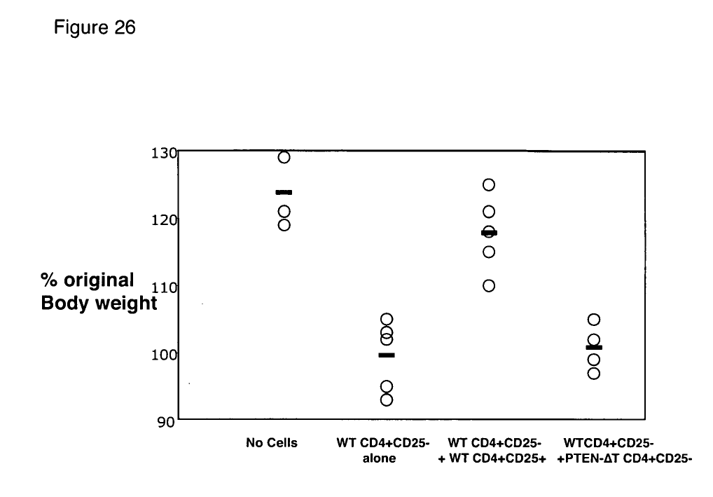

To examine whether PTEN activity negatively regulates IL-2 induced expansion of Tregs, CD4+CD25+CD45RB^{lo} cells from PTEN-ΔT mice were cultured in the presence of rIL-2 (100 U/ml) and assessed total cell numbers at various time points. As shown in FIG. 3A, viable PTEN-ΔT Treg cell numbers increased an average of 15-25 fold over a two week period in culture while no accumulation of wild-type Tregs, or CD4+CD25− T cells from either wild type or PTEN-ΔT mice was observed. CFSE dilution clearly illustrates the kinetics of cell division over the first 10 days in culture and cell proliferation was also assessed by incorporation of tritiated thymidine after 48 hours in culture with rIL-2 (FIGS. 3B&C). This expansion of PTEN-ΔT Tregs was due to the proliferative effects of IL-2R signaling as observed no difference in the levels of cell survival between wild-type and PTEN-ΔT Tregs (FIG. 25).

The proliferative response of PTEN-ΔT Tregs to IL-2 is dose-dependent, with cell division evident at as low as 10 U/ml of rIL-2 (FIG. 3D). This level of proliferation observed in PTEN-ΔT Tregs is less than that seen in activated CD4+ T cell blasts. In part, this may be due to endogenous IL-2 secretion by the activated CD4+ blasts, which increases the effective dose of IL-2 in each well. However it also suggests that in addition to PTEN, other parameters may also regulate IL-2 responsiveness of CD4+ T cells.

While not wishing to be bound by any one particular theory, the present data supports an explanation for PTEN-ΔT Treg expansion in response to IL-2 alone in which these cells may have already encountered TCR stimulation in vivo before isolation, thus facilitating their IL-2 responsiveness. However no differences in phosphorylation of ZAP-70 or Erk between freshly isolated wild-type and PTEN-ΔT Tregs were detected. Another consideration is that T cell developmental abnormalities which may occur in PTEN-ΔT mice could influence the behavior of CD4+CD25+ regulatory T cells. Although the mixed bone marrow chimera data indicates that Tregs develop normally in the absence of PTEN, to definitively exclude the possibility that potential developmental or homeostatic defects which may arise in PTEN-ΔT mice may alter the response of PTEN-ΔT Tregs to IL-2, the PTEN^{flox/flox} mice were crossed with Cre-ER transgenic mice in which Cre recombinase activity occurs only in the presence of the estrogen homolog Tamoxifen. Unlike PTEN-ΔT mice, no defects in peripheral or central tolerance were observed in these mice and the CD4+CD25+ regulatory T cell compartment was identical to that of Cre negative littermates. Culture of purified CD4+ T cells from Cre-ER/Pten^{flox/flox} mice in the presence of 1 nM 4-OH Tamoxifen (4-OHT) led to a significant decrease in the amount of PTEN expression after 72 hours (FIG. 17E). In addition, stimulation of Cre-ER/Pten^{flox/flox} CD4+CD25+ Tregs with IL-2 in the presence of 1 nM 4-OHT led to a robust proliferative response after 7 days in culture as assessed by CFSE dilution (FIG. 17F). Together these data confirm that PTEN deficient CD4+CD25+ regulatory T cells can be readily expanded upon IL-2R stimulation and that this response is not due to developmental defects, as a consequence of loss of PTEN.

PTEN Constrains IL-2 Responsiveness

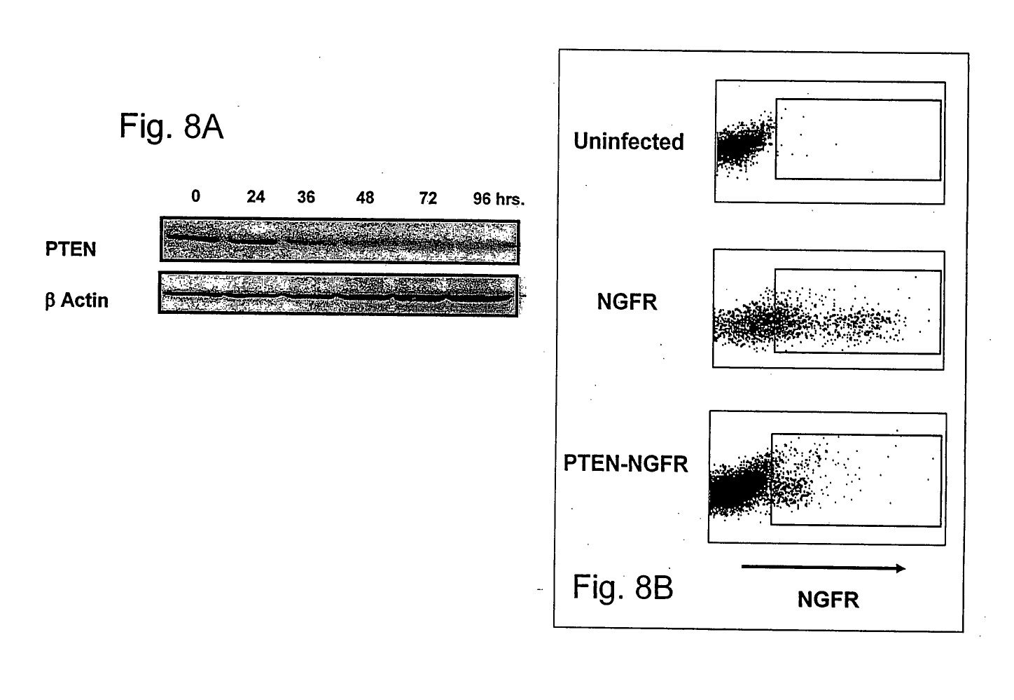

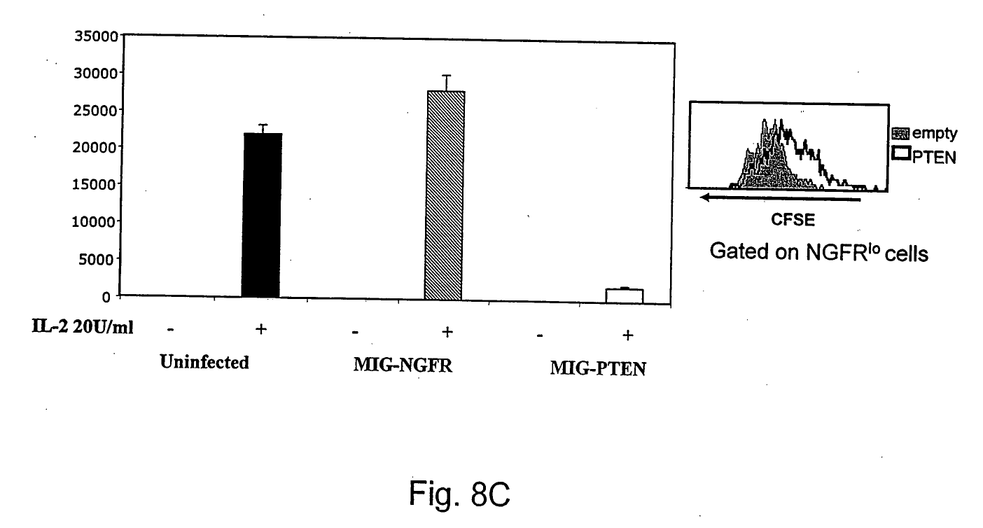

In order to assess the role of expression of PTEN in T cells, experiments were designed to introduce exogenous PTEN into the cells. T cells were isolated using methods disclosed elsewhere herein or otherwise using methods readily known in the art. T cells were transduced with a retroviral system. The results presented herein demonstrate that retroviral transduction of normal T cells with PTEN prevents the activation-induced downregulation of PTEN, and renders T cells unresponsive to IL-2 (FIG. 8).

Re-Expression of PTEN Blocks IL-2 Mediated Expansion of PTEN-ΔT Tregs

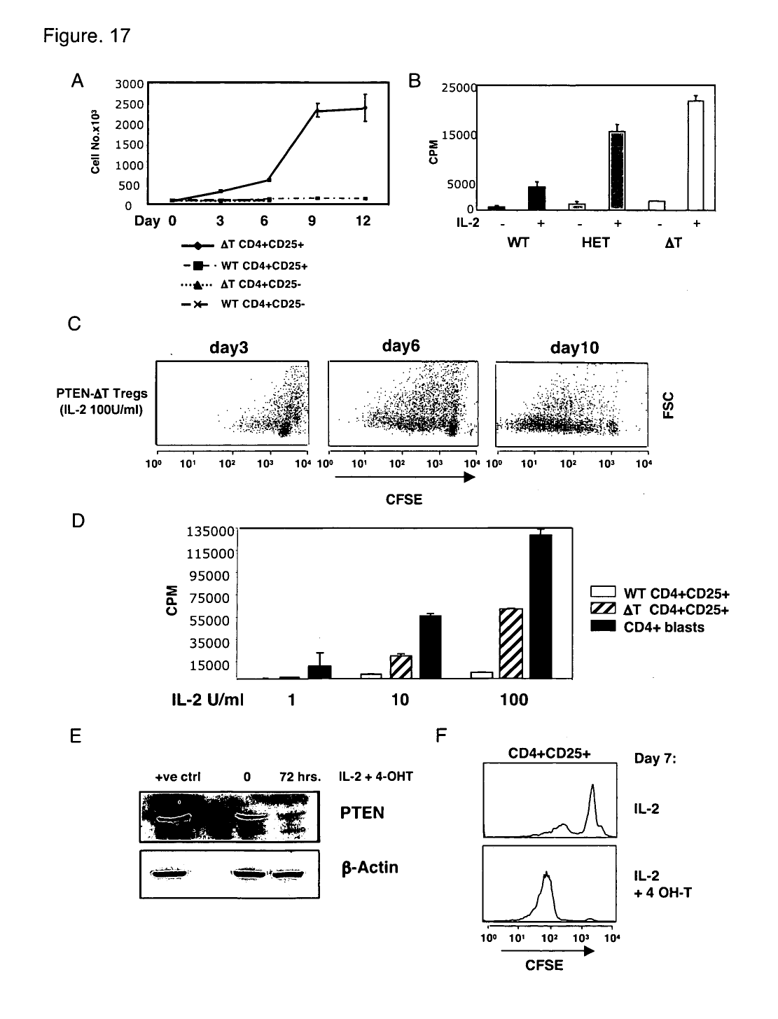

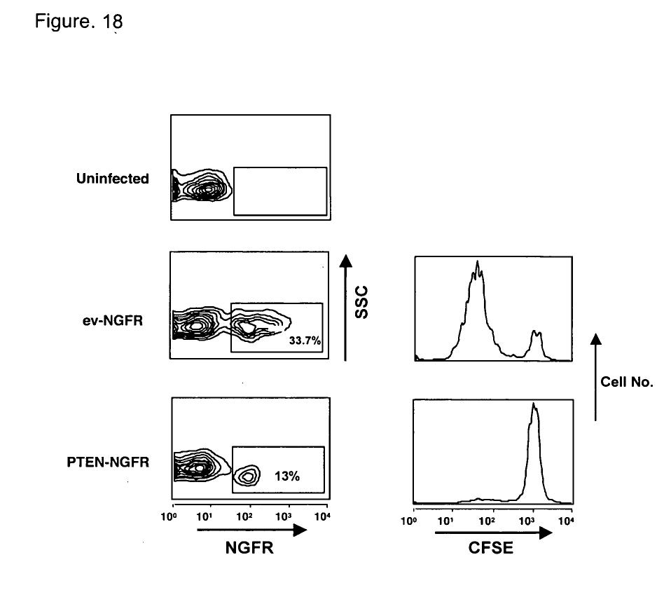

The gene for human Pten was cloned into a bicistronic retroviral vector also expressing the extracellular portion of the human NGFR as a marker of cell transduction. Transduction of CFSE labeled PTEN-ΔT Tregs with NGFR/MIGR1 empty vector typically resulted in 30-40% transduction efficiency as determined by flow cytometric analysis of NGFR expression. In contrast, the Pten containing retrovirus consistently resulted in 5-15% transduction efficiency (FIG. 18). Since efficient retroviral transduction requires target cells to be progressing through the cell cycle, the significantly lower efficiency of transduction observed with the PTEN containing virus is consistent with PTEN inhibiting cell cycle progression. After transduction, cells were cultured in the presence of rIL-2 (100 U/ml) for 4 days and levels of CFSE dilution of NGFR+ cells were examined. As shown in FIG. 18, although transduction of PTEN-ΔT Tregs with empty vector does not effect IL-2 induced proliferation, re-expression of PTEN results in complete inhibition of IL-2 mediated proliferation, restoring the hyporproliferative response observed in wild-type Tregs.

PTEN Deficient CD4+CD25+ Regulatory T Cells do not Proliferate in Response to TCR Stimulation

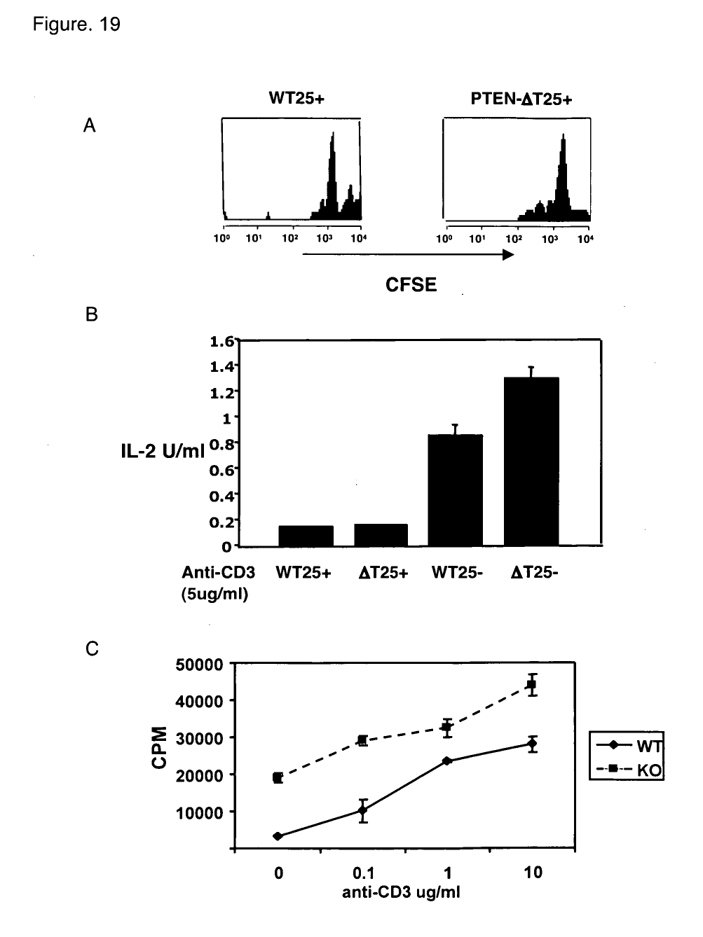

In addition to their hypoproliferative response to IL-2R stimulation, it has also been established that CD4+CD25+ regulatory T cells do not divide after stimulation with anti-CD3 antibody alone. This observation is most likely related to the relative inability of Tregs to produce IL-2. Similar to their wild type counterparts, stimulation of PTEN-ΔT Tregs with plate bound anti-CD3 did not induce proliferation nor any significant level of IL-2 production (FIGS. 19A-B). It has also previously been demonstrated that the hypoproliferative response of Tregs can be broken by TCR stimulation in the presence of a relatively high dose of IL-2. The response of PTEN-ΔT Tregs was examined upon stimulation with graded doses of anti-CD3 in the presence of rIL-2 (100 U/ml). PTEN-ΔT Tregs exhibited a more robust proliferative response in the presence of rIL-2 at all concentrations of anti-CD3 used in comparison to their wild-type counterparts (FIG. 19C). Importantly, the differences in proliferation observed at each concentration of anti-CD3 tested were equivalent to the difference seen in the presence of rIL-2 alone (i.e., the basal level of IL-2-induced proliferation in PTEN-ΔT Tregs). This suggests that while PTEN plays a significant role in regulating the IL-2 responsiveness of Tregs, the basal level of TCR responsiveness is unaltered in these cells.

Taken together these data confirm that Tregs can be expanded more readily in the absence of PTEN. However they also indicate that the hypoproliferative responses of Tregs to IL-2R or TCR stimulation alone are mediated, at least in part, through distinct mechanisms.

PTEN-ΔT Tregs Exhibit an Enhanced Rate of Peripheral Homeostatic Turnover In Vivo

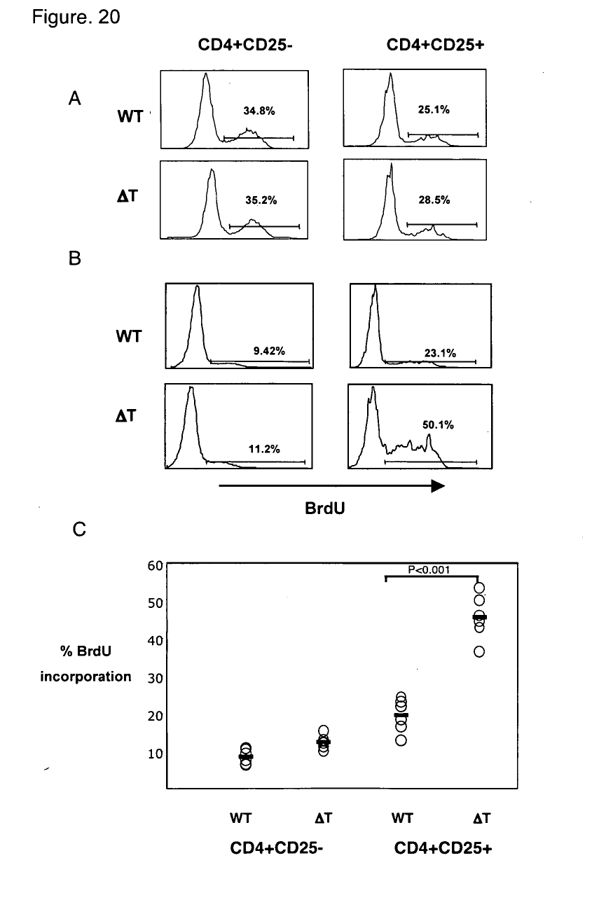

Although CD4+CD25+ Tregs are characterized by hypoproliferative responses in vitro, it is also clear that they readily undergo expansion in vivo. Furthermore, in unmanipulated animals Tregs in peripheral lymphoid organs exhibit higher levels of basal proliferation compared to their CD4+CD25− counterparts, and this response is known to depend, at least in part, on IL-2. Next, PTEN-ΔT mice and littermate controls were injected with 5-bromo-2′-deoxyuridine (BrdU) for three days, after which CD4+CD25+ and CD4+CD25− T cells from both the thymii and spleens of these mice were analyzed for BrdU incorporation. While similar levels of BrdU incorporation were seen in splenic CD4+CD25− cells from control versus PTEN-ΔT mice, splenic PTEN-ΔT Tregs exhibit a significantly higher rate of BrdU staining compared to wild-type CD4+CD25+ T cells (FIGS. 20B-C). Elevated BrdU uptake in splenic Tregs was a result of increased peripheral turnover, as no difference was found in the levels of BrdU incorporation in either single positive CD4CD25− or CD4CD25+ subsets from PTEN-ΔT mice compared to wild-type littermates (FIG. 6A). Together these observations demonstrate that PTEN plays a role in regulating the peripheral homeostasis of Tregs in vivo, with out any significant effects on Treg development in the thymus.

Deletion of PTEN in Tregs Facilitates Downstream Activation of PI-3Kinase Dependent Signaling Through the IL-2R

As discussed elsewhere herein, work on IL-2R signaling has shown that activation of both the JAK/STAT and PI-3 kinase (PI3K)/Akt pathways are critical for IL-2 induced proliferation. Also discussed elsewhere herein, activation of signaling pathways downstream of PI-3 kinase does not occur in Tregs in response to IL-2R stimulation and negative regulation of this signaling pathway by PTEN was identified as a possible mechanism for this observation.

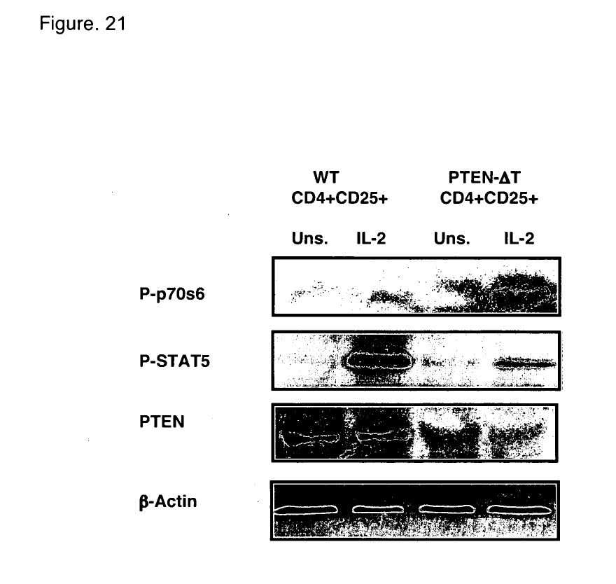

Next, freshly isolated Tregs from wild type mice, or Tregs isolated from PTEN-ΔT mice and expanded for 8 days with IL-2 (100 U/ml) to obtain necessary cell numbers were rested overnight in complete media then stimulated for 30 minutes with 100 U/ml rIL-2. Cell lysates were subsequently tested for activation of both JAK/STAT and PI-3 kinase dependent signaling pathways. As discussed elsewhere herein, stimulation of wild type Tregs resulted in a robust but isolated activation of JAK/STAT signaling, as shown by phosphorylation of STAT5, without any detectable activation of PI-3 kinase dependent signaling through phosphorylation of p70 S6 kinase. In contrast, while activation of JAK/STAT signaling was also clearly detectable after IL-2R stimulation of PTEN-ΔT Tregs, phosphorylation of p70 S6 kinase was observed, indicating that IL-2R stimulation activated PI-3kinase dependent signaling pathways in these cells (FIG. 21).

Overexpression of PTEN Inhibits IL-2 Mediated Expansion of Activated CD4+ T Cells

The results above suggest that PTEN is necessary and sufficient for the hypoproliferative response of Tregs to IL-2. Because IL-2 is known to enhance the proliferation of activated CD4+ T cells, it was next determined whether maintenance of its expression would have a similar inhibitory effect on IL-2R induced cell division in these cells.

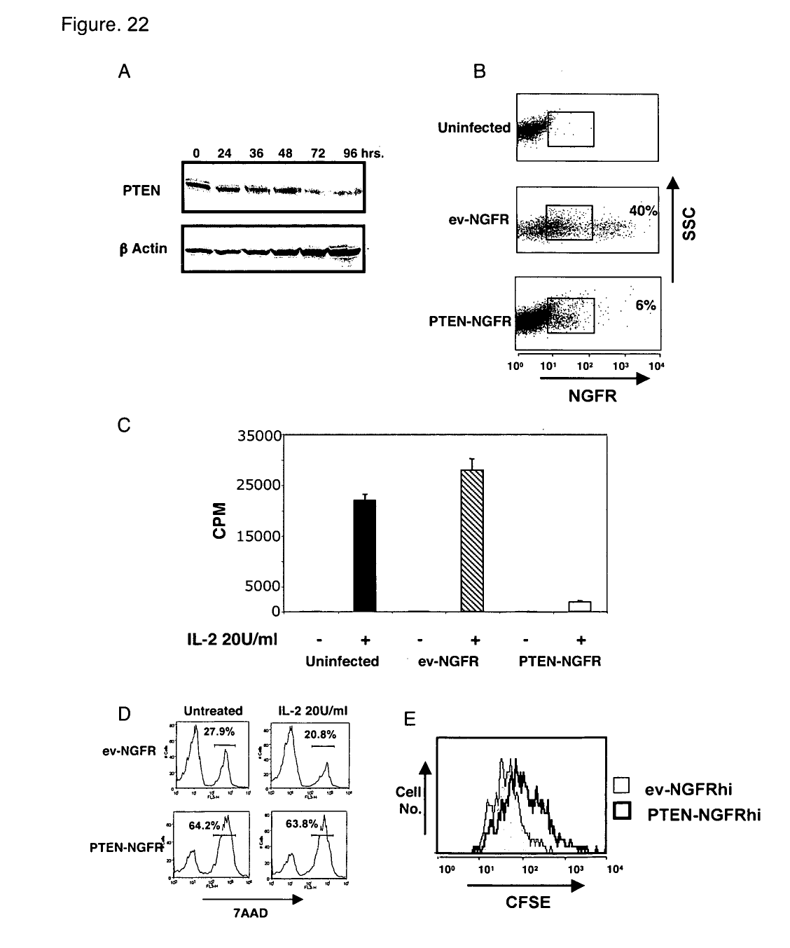

Activation of non-regulatory CD4+ T cells both induces CD25 expression and downregulates PTEN (FIG. 22A). To examine PTEN activity following T cell activation, retroviral transduction was used. Similar to PTEN-ΔT as discussed elsewhere herein, Tregs infection of activated CD4+ T cells with NGFR/MIGR1 empty vector typically resulted in 40-50% transduction efficiency while the Pten containing virus resulted in 5-10% transduction efficiency again consistent with PTEN inhibiting cell cycle progression (FIG. 22B). Purified NGFR positive cells from both mock (ev-NGFR) and PTEN (PTEN-NGFR) transduced cells, were subsequently cultured in the presence or absence of IL-2, and proliferation was assessed by thymidine incorporation. While cells expressing empty vector proliferated to a similar extent as non-transduced T cells, those expressing PTEN showed a dramatically decreased level of thymidine incorporation (FIG. 22C).

Previous studies, using ectopic expression of PTEN in T cell lines have supported a role for PTEN not only in inhibiting cell cycle progression but also in inducing apoptotic cell death. Using 7-AAD staining as a measure of cell death, it was found that PTEN overexpression leads to a significant increase in activated T cell death when compared to empty vector transduced cells (64% vs 28%). The addition of exogenous rIL-2 did not rescue this effect (FIG. 22D).

To exclude the possibility that cells overexpressing PTEN failed to incorporate thymidine exclusively due to cell death, CFSE dilution in live cells was measured as an alternative means to assess proliferation after PTEN transduction. Freshly isolated CD4+ T cells were CFSE labeled prior to activation with PMA and ionomycin for a period of 18 hours. These cells were then transduced with either empty or PTEN expressing virus before subsequent culture with rIL-2 (10 U/ml) for a further 48 hours. At this point live cells were examined, based on forward and side-scatter profiles, for NGFR expression, and CFSE dilution of NGFR positive subsets were compared. For comparison cells expressing identical levels of NGFR were analyzed. As shown in FIG. 22E, proliferation of PTEN overexpressing cells is significantly less than that observed in empty vector transduced cells, confirming that PTEN acts to inhibit IL-2 induced proliferation of activated CD4+ T cells. As an appropriate comparison, cells expressing identical levels of NGFR were analyzed (FIG. 22B), although the results described were identical even if all NGFR positive cells were included in the analysis. While the inhibition of IL-2 induced proliferation by PTEN in recently activated CD4+ cells is clear, it is far less significant than the almost complete inhibition observed in retrovirally transduced PTEN-ΔT Tregs (FIG. 18). This suggests that other factors in addition to PTEN may also regulate IL-2 mediated proliferation in activated non regulatory CD4⁺ T cells.

Expanded PTEN Deficient CD4+CD25+ Regulatory T Cells Retain their Suppressor Phenotype

One of the largest drawbacks to exploiting the potential of regulatory T cells in a therapeutic setting is the very low frequency with which these cells are found in normal healthy individuals. The results discussed herein demonstrate that in the absence of PTEN activity, these cells can be readily expanded ex vivo using only rIL-2. These data highlight the potential for targeting this lipid phosphatase to facilitate ex vivo expansion of Tregs in response to cytokine stimulation alone. However, such strategies could only be feasible if in vitro manipulation of these cells does not alter their regulatory potential. Therefore to determine whether PTEN deficient Tregs retain their regulatory phenotype after expansion with rIL-2, their ability to suppress the proliferation of wild type CD4+ responder T cells in vitro was examined, as well as their ability to suppress the development of inflammatory bowel disease in vivo.

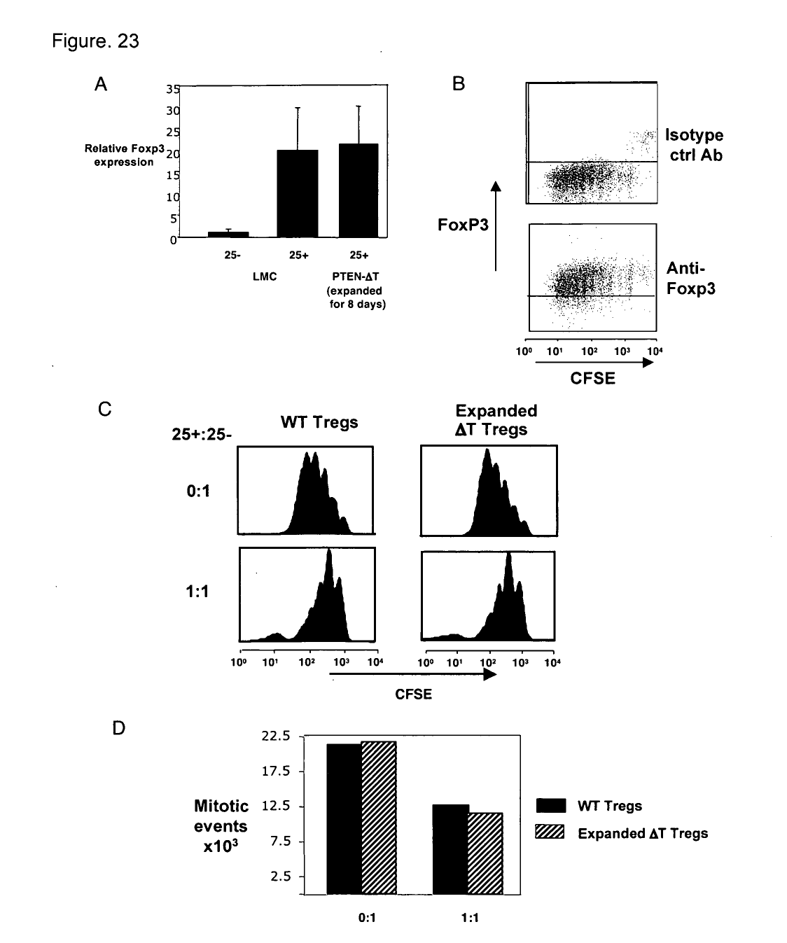

As shown in FIGS. 23A-B, Tregs expanded for eight days retain expression of Foxp3 mRNA and protein and their ability to inhibit CD4+ effector T cell proliferation. The level of suppression is similar to that observed with freshly isolated wild type Tregs (FIGS. 23C-D).

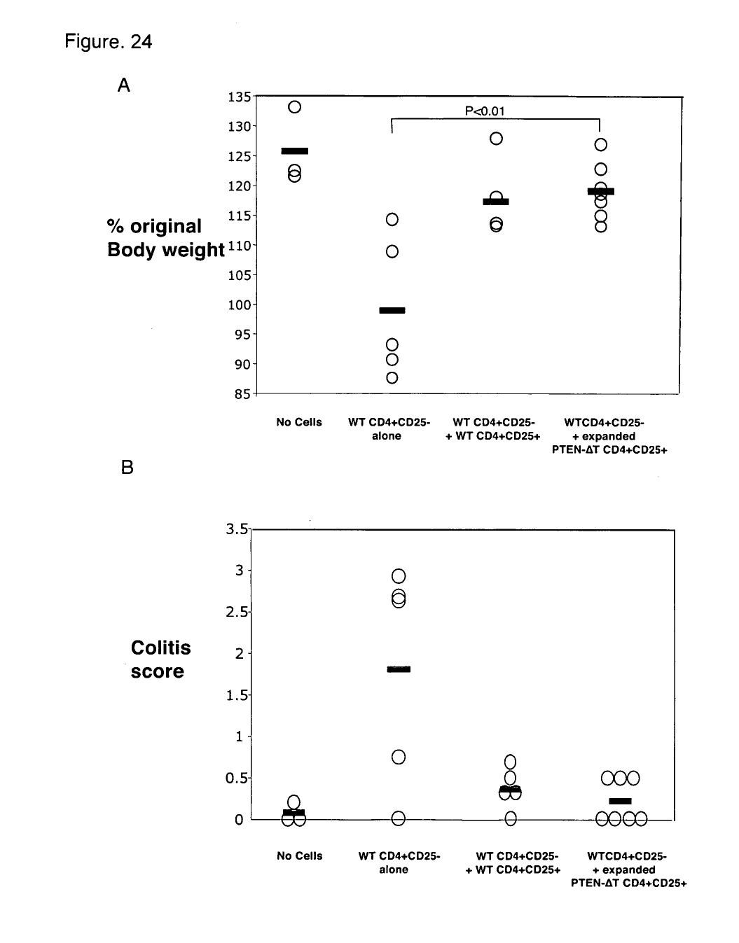

Adoptive transfer of CD4+CD25−CD45RB^{hi} cells into immunodeficient hosts has been demonstrated to result in the development of colitis, which can be prevented by co-transfer of regulatory T cells. To examine whether ex vivo expanded PTEN deficient Tregs could prevent disease in vivo, wild type CD4+CD25−CD45RB^{hi} cells were co-transferred with either wild type freshly isolated Tregs or PTEN-ΔT Tregs after expansion for 5 days in vitro with rIL-2. Similar to the above in vitro observations, expanded PTEN-ΔT Tregs can prevent the development colitis in vivo to the same extent as freshly isolated wild type Tregs (FIG. 24).

Prevention of colitis in this model may be mediated by cell competition for an existing niche in the host rather than through the direct suppressive activity of Tregs. If this were true in the present data, prevention of colitis may also occur if Tregs are replaced in this assay by PTEN-ΔT CD4+CD25− T cells. However, co-adoptive transfer of PTEN-ΔT non-regulatory T cells did not affect the development of colitis resulting from the transfer of wild-type CD4+CD25− T cells (FIG. 25).

Together these data confirm that CD4+CD25+ regulatory T cell development is intact in PTEN-ΔT mice and demonstrate that expansion of these cells with IL-2 in vitro does not alter their suppressor phenotype.

The data set forth herein also suggest that the breakdown in T cell tolerance observed in mice deficient in PTEN is the result of defects within non-regulatory T cell compartments. As described previously, PTEN deficient T cells are hyperesponsive to activation stimuli and less susceptible to AICD when compared to wild type controls.

Despite their apparently normal thymic development, PTEN-ΔT Tregs exhibit an increased rate of BrdU incorporation in the periphery (FIG. 20), and can be expanded quite readily in response to IL-2 in vitro (FIG. 17). Given these observations, and as IL-2 plays an important role in regulating the peripheral homeostasis of Tregs, it might be expected to see an increased number of peripheral Tregs in PTEN-ΔT mice. However the frequency of Tregs in PTEN-ΔT mice, before the onset of disease, is identical to that observed in littermate controls (FIG. 15). This indicates that in addition to exhibiting a higher level of peripheral homeostatic expansion, PTEN-ΔT Tregs also have a higher rate of turnover compared to their wild-type counterparts. These observations also suggest that the higher rate of turnover of CD4+CD25+ Tregs (compared with CD4+CD25− T cells) may be balanced by a higher rate of cell death perhaps due to the lack of available “space” in the periphery.

Arguably the greatest single barrier to harnessing the therapeutic potential of naturally occurring CD4+CD25+ Tregs for the treatment of immune disorders is their relative low frequency in normal healthy individuals as well as their “anergic” phenotype ex vivo. The data set forth herein indicates that by targeting PTEN activity, Tregs can be expanded in response to rIL-2 alone without the need to stimulate the TCR and co-stimulatory receptors. Most importantly, it is demonstrated that ex vivo expansion of PTEN deficient Tregs does not affect their regulatory phenotype, as illustrated by their ability to suppress the proliferation of effector T cells in vitro as well as their ability to prevent the development of colitis in vivo (FIGS. 21-22).

Taken together, the data set forth herein provides a mechanism for the hypoproliferative response of CD4+CD25+ Tregs to IL-2 in vitro and also identify PTEN as a negative regulator of peripheral Treg homeostasis in vivo. These data indicate that negative regulation of PTEN activity in concert with IL-2R stimulation may facilitate expansion of CD4+CD25+ Tregs both ex vivo and in vivo for potential therapeutic use.

PTEN Deficient CD4+ T Cells are Hyper-Responsive to TCR Stimulation

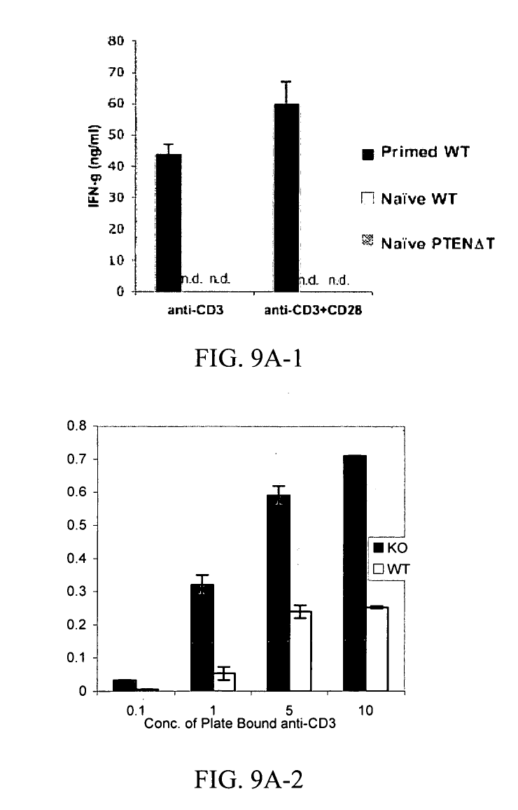

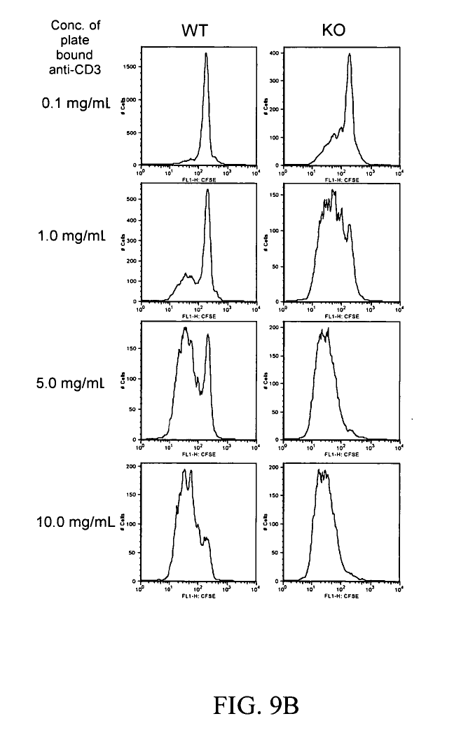

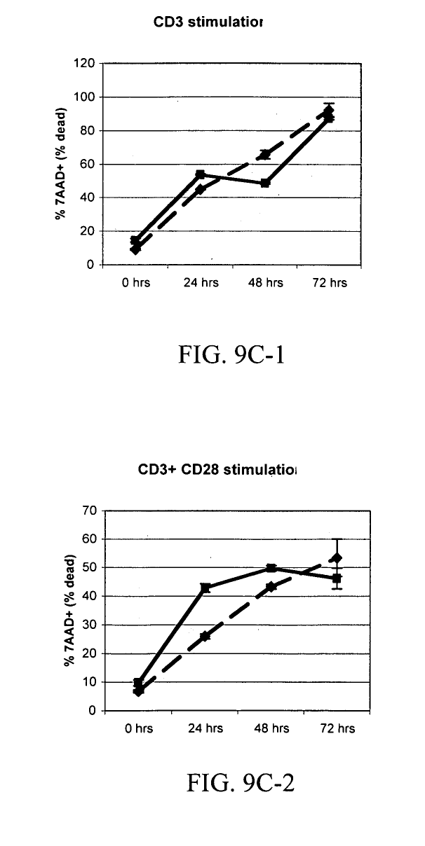

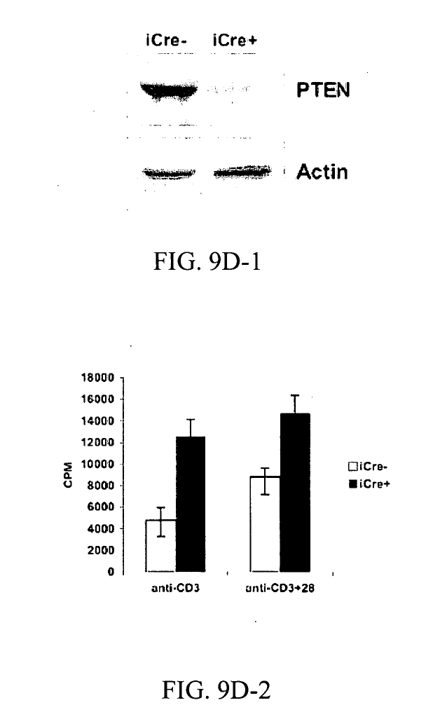

FIG. 9 demonstrates that PTEN deficient CD4+ T cells are hyper-responsive to TCR stimulation. To study the role of PTEN as a regulator of TCR signals, mice comprising a T cell specific deficiency in PTEN (PTENΔT) were generated by crossing PTEN^{flox/flox} mice with CD4-Cre transgenic mice. As previously demonstrated, mice with a deficiency of PTEN targeted to the T cell compartment develop both lymphomas and autoimmune disorders and die by approximately 17 weeks of age. The earliest sign of disease is an accumulation of activated CD4+ T cells in the periphery, evident by 6-8 weeks of age. To avoid contaminating the experiments with activated CD4+ T cells, all mice were used at 2-3 weeks of age. At this age, there are normal numbers and percentages of CD4+ T cells in both the spleen and lymph nodes of PTENΔT mice. Additionally, the percentage of CD4+ T cells that express the activation marker CD69 is similar in young PTENΔT and wild-type mice and CD4+ T cells isolated from young PTENΔT mice do not produce IFN-γ in response to primary stimulation in contrast with pre-activated (primed) T cells (FIG. 9A-1). Taken together, these results demonstrate that CD4+ T cells from 2-3 week old PTENΔT mice are phenotypically and functionally naïve.

FIG. 9A-2 demonstrates that CD4+ T cells from the lymph nodes and spleen of wild-type and PTEN-ΔT mice were stimulated with various concentrations of plate-bound anti-CD3 antibodies. Supernatants were collected and 24 hours and IL-2 production measured was by ELISA.

To determine the effect of PTEN deficiency on responsiveness to isolated TCR signals, CD4+ T cells were stimulated with plate-bound anti-CD3. PTENΔT CD4+ T cells produced far greater amounts of IL-2 and proliferated more extensively than wild-type cells at all concentrations of anti-CD3 stimulation, although the differences were most prominent at limiting doses. To deliver TCR signals alone in a more physiologic setting, cells were also stimulated with soluble anti-CD3 in the presence of APCs using CTLA4Ig to block CD28 engagement. IL-2 production and proliferation by PTENΔT CD4+T cells were similarly enhanced under these alternate conditions. FIG. 9B depicts CD4+ T cells that were isolated and stimulated as described above, and then harvested after 72 hours with proliferation measured by the dilution of CFSE dye assay.

Previous work demonstrates that PTENΔT CD4+ T cells have enhanced survival in response to certain apoptotic stimuli, including anti-Fas antibodies and γ irradiation. However, no difference was found in viability between wild-type and PTEN deficient CD4+ T cells in response to anti-CD3 stimulation alone or in combination with CD28 signals. These data suggest that the enhanced responsiveness of PTENΔT T cells to TCR signals is not simply a result of enhanced survival.

FIG. 9C-1 depicts iCre expressing and non-expressing mice that were injected i.p. for six consecutive days with tamoxifen (1 mg). Two days following the final injection, CD4+ T cells were purified from the lymph nodes. Cells were stimulated for 72 hours with plate-bound anti-CD3 alone or in combination with anti-CD28. ³H-thymidine was added to the wells for the final 16 hours of the stimulation.

To determine if altered T cell development contributed to the phenotype of PTENΔT T cells, mice were generated in which PTEN could be conditionally deleted within the mature T cell compartment by crossing PTEN^{flox/flox} mice with ER-Cre transgenic mice. Once generated, both ER-Cre expressing and non-expressing mice were injected with tamoxifen for six consecutive days. This treatment was sufficient to greatly reduce PTEN protein levels in the CD4 T cell compartment of mice expressing the ER8 Cre transgene (FIG. 9C2). In accordance with the response of PTENΔT CD4+ T cells from young mice, induced deletion of PTEN in mature CD4+ T cells resulted in an enhanced proliferative response to TCR signals either alone or in the presence of costimulation (FIG. 9C-1). These data suggest that altered development of T cells does not account for the enhanced responsiveness of PTEN deficient T cells.

FIGS. 9D-1 and 9D-2 depict wild-type or PTEN deficient T cells that were stimulated as described for FIG. 9C and then viability was measured at various time points using 7AAD.

Hyper-Activation of the PI3K Pathway is Observed in PTENΔT CD4+ T Cells Stimulated Through the TCR and is Required for Enhanced Cytokine Production

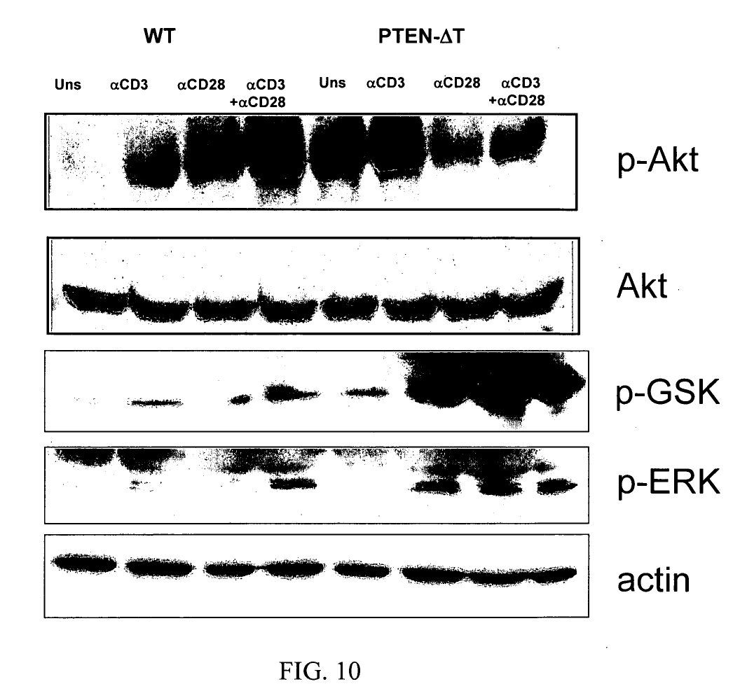

FIG. 10 demonstrates that PTEN deficiency results in hyperactivation of the PI3K pathway in response to TCR signals. CD4+ T cells were isolated from the spleen and lymph nodes of wild-type and PTEN-ΔT mice and were either left unstimulated, or stimulated with anti-CD3, anti-CD28, or a combination of the two antibodies for 30 minutes. Cell lysates were prepared and probed with phosphospecific antibodies for Akt, GSK, and ERK. Membranes were re-probed with Akt or actin as loading controls. Wild-type T cells fail to significantly activate PI3K in response to anti-CD3 stimulation alone, as evidenced by only a minimal induction of phosphorylated PI3K targets, such as Akt and GSK (FIG. 10A). In contrast, there is enhanced phosphorylation of PI3K targets in PTENΔT T cells receiving TCR stimulation. These data suggest that PTEN deficiency allows T cells to hyper-activate PI3K in response to TCR signals alone.

To test if PI3K activation is required for augmented IL-2 production by PTENΔT T cells, wild-type and PTENΔT CD4+ T cells were pre-incubated with chemical inhibitors to block either the activation of PI3K (LY294002) or Akt (Akt Inhibitor V). Following incubation with either inhibitor, the cells were stimulated as described previously with plate-bound anti-CD3. Using either inhibitor, the activation of a PI3K target Akt was modulated without disrupting the activation of ERK, a protein that can be activated independently of PI3K (FIG. 10C), or interfering with cell viability (as measured by 7AAD staining at the end of the stimulation period).

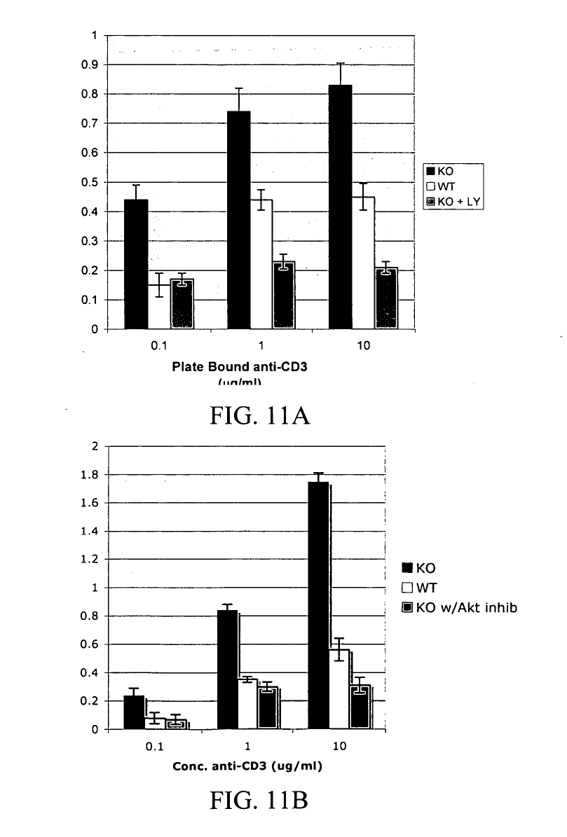

FIG. 11 demonstrates that activation of both PI3K and Akt is required for enhanced responsiveness of PTEN deficient T cells. CD4+ T cells were pre-incubated with either LY294002 (FIG. 11A) to inhibit PI3K activation, Triciribine (FIG. 11B) to inhibit Akt activity, or were allowed to rest in complete media for 30 minutes in the absence of chemical inhibitors. This incubation was followed by stimulation with various concentrations of anti-CD3. After 24 hours, supernatants were collected and IL-2 production was measured by ELISA.

In the presence of either the PI3K or the Akt inhibitor, IL-2 production was abrogated in the PTENΔT CD4+ T cells stimulated through the TCR (FIGS. 11C and 11D). Taken together, these data indicate that PTENΔT CD4+ T cells require both PI3K and Akt to produce augmented levels of IL-2 in response to TCR stimulation.

CD4+ T Cells that Lack PTEN have a Diminished Requirement for Costimulation

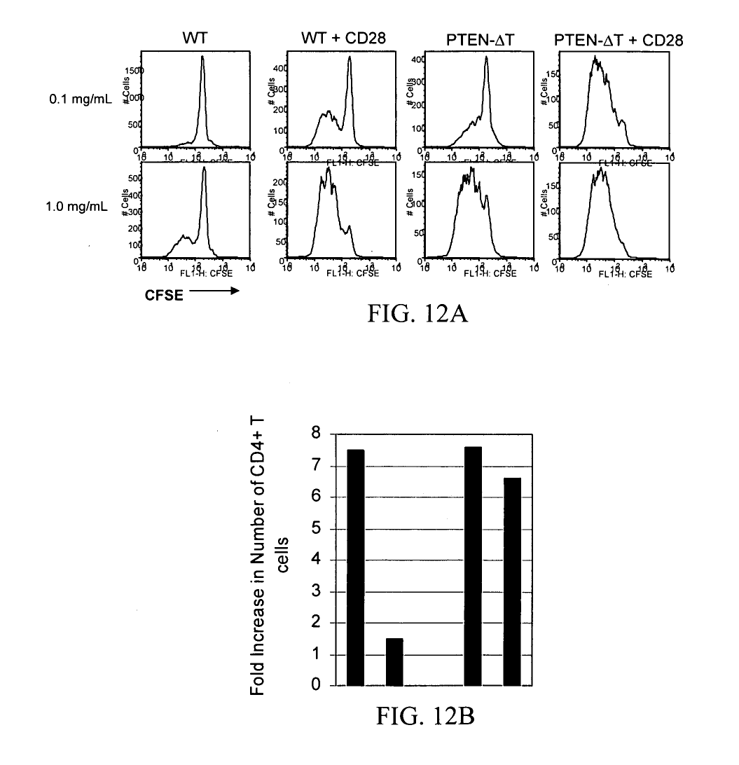

FIG. 12 demonstrates that CD4+ T cells that lack PTEN have a diminished requirement for costimulation. FIG. 12A depicts CD4+ T cells that were CFSE labeled and stimulated with various concentrations of anti-CD3 alone or in combination with anti-CD28. After 72 hours, cells were harvested and proliferation measured by dilution of the CFSE dye. FIG. 12B depicts results based on injection of either wild-type or PTENΔT mice (B6 background) with allogeneic (BALB/c) splenocytes and enumerated cells in the draining popliteal lymph node 72 hours later. Consistent with prior reports, the approximately 10-fold expansion observed in response to alloantigen immunization is almost completely dependent on CD28 signals, being blocked by treatment of mice with CTLA4Ig. In contrast, the expansion/accumulation of PTEN deficient CD4+ T cells is virtually unaffected by a lack of CD28 signals. These data further support the data herein demonstrating that PTEN deficiency relieves the requirement for CD28 costimulation. Similar results were obtained using wild type and PTEN-ΔT mice (C57BL/6 background) that were injected in the footpad with 20×10⁶ Balb/c splenocytes. Half of the mice also received an i.p. injection of CTLA4Ig (250 μg). Five days later, the draining and non-draining lymph nodes were harvested and the number of CD4+ T cells were quantified using flow cytometry.

CD4+ T Cells that Lack PTEN are Responsive to Treg-Mediated Suppression

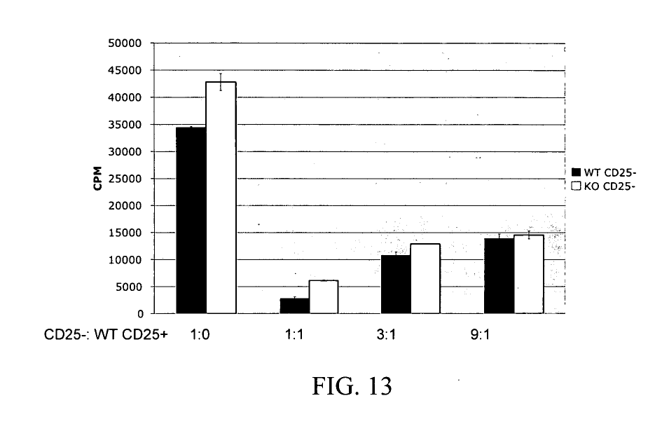

FIG. 13 demonstrates that CD4+ T cells that lack PTEN are still responsive to Treg mediated suppression. Wild-type Tregs (CD4+CD25+CD45RB^{lo}) were co-cultured at the indicated ratios with either wild type or PTEN deficient responder cells (CD4+CD25−CD45RB^{hi}). Cells were stimulated in the presence of irradiated APCs with soluble anti-CD3 (0.5 μg/ml) for 72 hours. ³H-thymidine was added to the wells for the final 16 hours of the stimulation.

PTEN Regulates Anergy Induction In Vitro and In Vivo

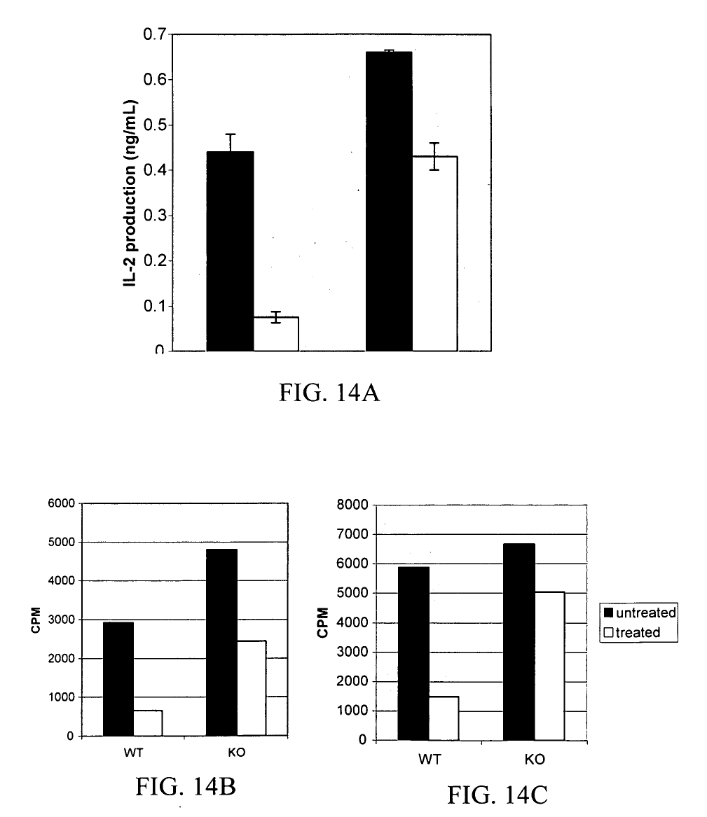

FIG. 14 demonstrates that PTEN regulates anergy induction both in vitro and in vivo. FIG. 14A depicts wild type and PTEN-ΔT CD4+ T cells that were stimulated with soluble anti-CD3 (1 μg/ml) in the presence of APCs (3:1 APC to T cell ratio), either with or without the addition of CTLA4Ig (10 μg/ml) to block CD28 costimulation. After 3 days of stimulation, the cells were washed and rested in complete media for 24 hours. Following this rest, all cells were restimulated with anti-CD3 (1 μg/ml) and anti-CD28 (5 μg/ml) coated beads. After 24 hours of secondary stimulation, supernatants were collection and IL-2 production was measured by ELISA. FIG. 14B depicts wild type and PTEN-ΔT mice that were injected i.v. with SEB (50 μg). Seven days later, spleens were harvested and Vβ8+CD4+ T cells were isolated and restimulated in vitro for 72 hours with various concentrations of SEB. ³H-thymidine was added to the wells for the final 16 hours of the stimulation.

For in vitro anergy induction, wild-type or PTEN deficient CD4+ T cells were stimulated with soluble anti-CD3 and APCs either in the absence or presence of CTLA4Ig for 72 hours, rested for 24 hours, restimulated with anti-CD3 and anti-CD28 coated beads and IL-2 levels measured. In contrast to wild-type cells, which are unable to produce significant quantities of IL-2 following initial stimulation under anergizing conditions, CD28 blockade did not induce a state of unresponsiveness in PTEN deficient T cells. In fact, PTENΔT CD4+ T cells initially subjected to anergizing conditions produced as much IL-2 upon restimulation as pre-activated wild-type CD4+ T cells. To test susceptibility to anergy induction in vivo, a model of superantigen induced tolerance was used. In vivo treatment of mice with superantigen results in a massive expansion of Vβ specific T cells, followed by a contraction phase. Those Vβ specific T cells that remain in the host following contraction are anergic, and consequently do not respond to restimulation with superantigen. Previous studies have shown that peripheral deletion is defective in PTENΔT mice following SEB-induced expansion. However, the functionality of the PTEN deficient cells that remain in the host is not known.

To determine if PTEN deficient CD4+ T cells are susceptible to superantigen induced tolerance, both wild-type and PTENΔT mice were injected with staphylococcal enterotoxin B (SEB). On day seven following treatment, Vβ8 specific CD4+ T were harvested and restimulated in vitro with SEB. Vβ8+ CD4+ T cells isolated from SEB treated wild-type mice exhibit a reduced proliferative response to restimulation with SEB compared to the same population of cells isolated from untreated wild-type mice. In contrast, PTEN deficient CD4+ Vβ8+ T cells from SEB-treated mice proliferated robustly in response restimulation. Thus, loss of PTEN in CD4+ T cells can prevent appropriate induction of anergy in vivo.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A PTEN-deficient T cell, wherein said T cell is selected from the group consisting of a cytotoxic T cell, a helper T cell, and a regulatory T cell.

2. The cell of claim 1, wherein PTEN in said T cell is mutated.

3. The cell of claim 1, wherein PTEN in said T cell is deleted.

4. The cell of claim 1, wherein expression of PTEN in said T cell is inhibited using an inhibitor selected from the group consisting of a small interfering RNA (siRNA), a microRNA, an antisense nucleic acid, a ribozyme, an expression vector encoding a transdominant negative mutant, an intracellular antibody, a peptide and a small molecule.

5. The cell of claim 4, wherein said inhibitor is an siRNA.

6. The cell of claim 5, wherein said siRNA is selected from the group consisting of a double stranded oligonucleotide, a single stranded oligonucleotide, and a polynucleotide.

7. The cell of claim 5, wherein said siRNA is chemically synthesized.

8. The cell of claim 4, wherein said inhibitor further comprises a physiologically acceptable carrier.

9. The cell of claim 8, wherein said physiologically acceptable carrier is a liposome.

10. The cell of claim 4, wherein said inhibitor is encoded by an isolated polynucleotide cloned into an expression vector.

11. The cell of claim 10, wherein said expression vector is selected from the group consisting of a plasmid DNA, a viral vector, and a mammalian vector.

12. The cell of claim 10, wherein said expression vector further comprises an integration signal sequence which facilitates integration of said isolated polynucleotide into the genome of a host cell.

13. The cell of claim 1, wherein said cell is capable of regulating an immune response.

14. The cell of claim 13, wherein said immune response is associated with a disease selected from the group consisting of an infectious disease, a cancer, an autoimmune disease, graft rejection, atherosclerosis, and graft versus host disease (GVHD).

15. The cell of claim 1, wherein said cell exhibits an increased proliferation rate compared to an otherwise identical functional PTEN T cell.

16. A genetically modified T cell expressing an increased level of a PTEN polynucleotide compared with an otherwise identical T cell not so genetically modified.

17. The cell of claim 16, wherein said T cell is selection from the group consisting of a cytotoxic T cell, a helper T cell, and a regulatory T cell.

18. The cell claim 16 wherein, the PTEN polynucleotide is expressed from a vector selected from the group consisting of a plasmid DNA, a viral vector, and a mammalian vector.

19. A method of regulating T cell cellular proliferation, the method comprising modulating PTEN expression and/or activity in said T cell by adding to the cell a composition that regulates the expression and or activity of PTEN.

20. The method of claim 19, wherein said T cell is selection from the group consisting of a cytotoxic T cell, a helper T cell, and a regulatory T cell.

21. The method of claim 19, wherein expression of a PTEN polynucleotide in said T cell is down-regulated.

22. The method of claim 19, wherein expression of a PTEN polynucleotide in said T cell is increased.

23. A method of regulating an immune response, the method comprising modulating PTEN in said T cell by adding to the cell a composition that regulates the expression and or activity of PTEN.

24. The method of claim 23, wherein said T cell is selection from the group consisting of a cytotoxic T cell, a helper T cell, and a regulatory T cell.

25. The method of claim 23 wherein, expression of a PTEN polynucleotide in said T cell is down-regulated.

26. The method of claim 23, wherein expression of a PTEN polynucleotide in said T cell is increased.

27. A method of treating a disease or disorder in a patient, wherein said disease or disorder selected from the group consisting of an infectious disease, a cancer, an autoimmune disease, graft rejection, atherosclerosis, and graft versus host disease (GVHD), the method comprising administering to said patient an isolated PTEN-deficient T cell.

28. The method of claim 27, wherein said T cell is selected from the group consisting of a cytotoxic T cell, a helper T cell, and a regulatory T cell.

29. The method of claim 27, wherein said T cell is cultured in vitro prior to administering to said patient in need thereof.

30. The method of claim 29, wherein said T cell is cultured in vitro in the presence of IL-2.

31. A method of treating a disease or disorder selected from the group consisting of an infectious disease, a cancer, an autoimmune disease, graft rejection, atherosclerosis, and graft versus host disease (GVHD), the method comprising administering a composition comprising an inhibitor of PTEN to a patient in need thereof, wherein said inhibitor is selected from the group consisting of a small interfering RNA (siRNA), a microRNA, an antisense nucleic acid, a ribozyme, an expression vector encoding a transdominant negative mutant, an intracellular antibody, a peptide and a small molecule.

32. A method of treating a disease or disorder in a patient, wherein said disease or disorder is selected from the group consisting of an infectious disease, a cancer, an autoimmune disease, graft rejection, atherosclerosis, and graft versus host disease (GVHD), the method comprising administering an isolated T cell so modified to prevent downregulation of PTEN to said patient.

33. The method of claim 32, wherein said T cell is selected from the group consisting of a cytotoxic T cell, a helper T cell, and a regulatory T cell.

34. The method of claim 32, wherein said T cell is cultured in vitro prior to administering to said patient.

35. A method of treating a disease or disorder in a patient, wherein said disease or disorder is selected from the group consisting of an infectious disease, a cancer, an autoimmune disease, graft rejection, atherosclerosis, and graft versus host disease (GVHD), the method comprising administering a composition comprising a polynucleotide encoding PTEN to said patient.

36. A method of increasing the activity of a CD4 T cell, said method comprising inhibiting PTEN in said CD4 T cell.

37. The method of claim 36, wherein PTEN in said T cell is mutated.

38. The method of claim 36, wherein PTEN in said T cell is deleted.

39. The method of claim 36, wherein PTEN in said CD4 T cell is inhibited using an inhibitor selected from the group consisting of a small interfering RNA (siRNA), a microRNA, an antisense nucleic acid, a ribozyme, an expression vector encoding a transdominant negative mutant, an intracellular antibody, an antibody, a peptide and a small molecule.

40. The method of claim 39, wherein said inhibitor is an siRNA.

41. The method of claim 40, wherein said siRNA is selected from the group consisting of a double stranded oligonucleotide, a single stranded oligonucleotide, and a polynucleotide.

42. The method of claim 40, wherein said siRNA is chemically synthesized.

43. The method of claim 40, wherein said inhibitor is a small molecule.

44. A method of preventing anergy in a CD4 T cell, said method comprising inhibiting PTEN in said CD4 T cell.

45. The method of claim 44, wherein PTEN in said T cell is mutated.

46. The method of claim 44, wherein PTEN in said T cell is deleted.

47. The method of claim 44, wherein PTEN in said CD4 T cell is inhibited using an inhibitor selected from the group consisting of a small interfering RNA (siRNA), a microRNA, an antisense nucleic acid, a ribozyme, an expression vector encoding a transdominant negative mutant, an intracellular antibody, an antibody, a peptide and a small molecule.

48. The method of claim 47, wherein said inhibitor is an siRNA.

49. The method of claim 48, wherein said siRNA is selected from the group consisting of a double stranded oligonucleotide, a single stranded oligonucleotide, and a polynucleotide.

50. The method of claim 47, wherein said siRNA is chemically synthesized.

51. The method of claim 47, wherein said inhibitor is a small molecule.
